(19) United States
(12) Patent Application Publication
(10) Pub. No.: US 2009/0285888 A1
(43) Pub. Date: Nov. 19, 2009

(54) DERIVATIVES OF DIHYDROXYPHENYLALANINE

(76) Inventor: Gisela Susilo, Koln (DE)

Correspondence Address: J C PATENTS, 4 VENTURE, SUITE 250, IRVINE, CA, 92618, US

(21) Appl. No.: 11/914,207

(22) PCT Filed: May 14, 2006
(86) PCT No.: PCT/DE06/00830

§ 371 Date: Aug. 22, 2008

(51) Int. Cl. A61K 9/28; C07D 339/04; A61K 31/385; A61K 31/235; C07C 233/49; C07C 69/773; A61P 25/28

(52) U.S. Cl. 424/474; 549/39; 514/440; 514/548; 554/63; 560/73; 514/533

(57) ABSTRACT

The invention relates to derivatives of dihydroxyphenylalanine, to their synthesis and to pharmaceutical compositions containing said derivatives of dihydroxyphenylalanine. Furthermore, the invention relates to the use of said derivatives of dihydroxyphenylalanine and said pharmaceutical compositions for the treatment and prophylaxis of movement disorders, neurodegenerative diseases, Alzheimer's disease, Parkinson's disease, hemiparkinson-hemiatrophy, parkinsonian syndrome, Lewy body disease, frontotemporal dementia, Lytico-Bodig disease (parkinsonism/dementia/amyotrophic lateral sclerosis), striatonigral degeneration, Shy-Drager syndrome, sporadic olivopontocerebellar degeneration, progressive atrophy of the globus pallidus, progressive supranuclear palsy, Hallervorden-Spatz disease, Huntington's disease, X-linked dystonia-parkinsonism (Lubag), mitochondrial cytopathy with striatal necrosis, neuroacanthocytosis, restless legs syndrome, Wilson's disease.

The invention relates to derivatives of dihydroxyphenylalanine, methods for their synthesis as well as to the use thereof and to pharmaceutical compositions comprising these derivatives of dihydroxyphenylalanine.

DOPA is known under the IUPAC name 2-amino-3-(3,4-dihydroxyphenyl)-propionic acid and under the trivial name Levodopa and is used in particular for the treatment of Parkinson's disease.

Parkinson's disease, also known as Parkinson's syndrome, is one of the chronic diseases which are still incurable. The course of the disease is characterized in that the nerve cells which do not contain the chemical messenger dopamine slowly die in the substantia nigra of the brain (the black substance). Consequently, the formation of the chemical messenger dopamine in sufficient quantities is not ensured. Mutations (e.g. Lewy bodies) can also be found in other parts of the brain such as the nucleus coeruleus, the raphe nuclei, the nucleus basalis of meynert, the nucleus of the vagus and the hippocampus. Dopamine is an essential messenger for the control of the musculoskeletal system and a lack of dopamine causes movement disorders such as trembling (resting tremor), involuntary muscle tensions (rigidity) and a slowness of movement (hypokinesia). In the advanced stage further movement disorders will appear such as the inability to commence a movement (freezing) and the impossibility of maintaining a straight posture associated with a high risk of falls. Furthermore, thought processes and memory are affected as well as emotions, with onset of depression and, in the final stage, dementia.

Parkinson's disease is divided into a sporadic form (95% of the persons concerned) and a familial form. The latter form is mainly caused due to hereditary transmission of the risk of disease. Moreover, several diseases involving movement disorders are described; their appearance, however is due to other causes. They are referred to as secondary parkinsonism. These forms may be caused by pharmaceuticals such as neuroleptics and reserpine and derivatives thereof. Furthermore, a hemiparkinson-hemiatrophy syndrome is known. A Parkinson syndrome can also be associated with hydrocephalus (hydrocephaly), oxygen deficiency, infections of the brain (encephalitis), manganese poisoning, carbon monoxide (CO) poisoning, 1-methyl-4-phenyl-1,2,3,6-tetrahydropyridine (MPTP) poisoning and cyanide poisoning. Further causes are parathyroid diseases, brain tumor, brain lesion, and multiple occlusions (infarctions) of brain vessels. Further diseases with movement disorders are Alzheimer's disease, diffuse Lewy body disease, frontotemporal dementia, Lytico-Bodig disease (parkinsonism/dementia/amyotrophic lateral sclerosis), striatonigral degeneration, Shy-Drager syndrome, sporadic olivopontocerebellar degeneration, progressive atrophy of the globus pallidus, progressive supranuclear palsy, Hallervorden-Spatz disease, Huntington's disease, X-linked dystonia-parkinsonism (Lubag), mitochondrial cytopathy with striatal necrosis, neuroacanthocytosis and Wilson's disease.

Initially, L-DOPA was used as a promising medicament, but soon side effects of long-term therapies were observed which ranged from dyskinesias (abnormal, involuntary movements) and dystonias (painful muscle cramps) to abruptly alternating phases of moving and freezing. Furthermore, is was found that L-DOPA may promote the destruction of dopamine-containing nerve cells in the brain.

In Germany, 1 to 2% of the population over sixty years of age suffer from Parkinson's disease. Consequently, there is an urgent need to provide medicaments which are suitable for the treatment of Parkinson's disease and other movement disorders.

It is the object of the present invention to provide physiologically acceptable substances and pharmaceutical compositions which can be used, amongst others, for the treatment of movement disorders.

This object is solved by the technical teaching of the independent claims. Further advantageous embodiments, aspects, and details of the invention are evident from the dependent claims, the description and the examples.

The invention relates to compounds of the general formula (I)

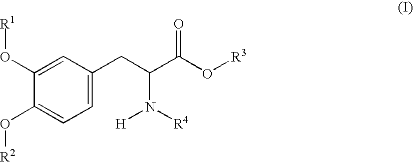

wherein
R¹ and R² represent, independently of each other, the following residues: —H, —R⁸, —R⁹, —CO—H, —CO—CH₃, —CO—C₂H₅, —CO—C₃H₇, —CO—C₄H₉, —CO—C₅H₁₁, —CO—C₆H₁₃, —CO—CH(CH₃)₂, —CO-cyclo-C₃H₅, —CO—CH₂—CH(CH₃)₂, —CO—CH(CH₃)—C₂H₅, —CO—C(CH₃)₃, —CO-cyclo-C₄H₇, —CO-cyclo-C₅H₉, —CO-cyclo-C₆H₁₁, —C≡CH, —C≡C—CH₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CF₃, —C₂F₅, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —CH₂—CH═CH—CH₃, —CH═CH₂, —CH₂—CH═CH₂, —CH═CH—CH₃, -cyclo-C₃H₅, -cyclo-C₄H₇, -cyclo-C₅H₉, -cyclo-C₆H₁₁,

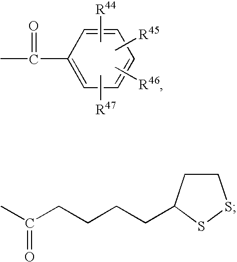

R³ represents a residue —CH₂CH₂O—R⁵, —H, —C≡CH, —C≡C—CH₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄C(CH₃)₃,
—CH(CH₃)—C(CH₃)₃, —CH═CH₂, —CH₂—CH═CH₂, —C(CH₃)═CH₂, —CH═CH—CH₃, —C₂H₄—CH═CH₂, —CH₂—CH═CH—CH₃, —CH═CH₂, —CH₂—CH═CH₂, —CF₃, —C₂F₅, —CH═CH—CH₃, -cyclo-C₃H₅, -cyclo-C₄H₇, -cyclo-C₅H₉, -cyclo-C₆H₁₁;
R⁴ and R⁵ represent, independently of each other, a group —CO—R⁶ or —CO—R⁷ or —H, wherein R³ and R⁴ do not at the same time represent —H; and
R⁶ and R⁷ represent, independently of each other, the following residues: —R¹⁰, —R¹¹, a linear saturated alkyl chain with 2-25 carbon atoms, a branched saturated alkyl chain with 2-25 carbon atoms, a branched or unbranched alkenyl chain with 2-25 carbon atoms, a branched or unbranched alkinyl chain with 2-25 carbon atoms, a polyunsaturated branched or unbranched alkenyl chain with 2-25 carbon atoms, a polyunsaturated branched or unbranched alkinyl chain with 2-25 carbon atoms, a polyunsaturated branched or unbranched alkeninyl chain with 2-25 carbon atoms, a branched or unbranched alkyl chain with 2-25 carbon atoms comprising a carbocycle or a heterocycle, a branched or unbranched alkyl chain with 2-25 carbon atoms comprising one or more hydroxy groups, alkoxy groups, thio groups, mercapto groups, amino groups, halogen groups, carbonyl groups, carboxyl groups and/or nitro groups;
R⁸, R⁹, R¹⁰ and R¹¹ represent, independently of each other, the following residues: —CH₂R¹², —CHR¹³R¹⁴, —CR¹⁵R¹⁶R¹⁷, —CH₂—CR¹⁸R¹⁹R²⁰, —CH₂—CHR²¹R²², —CR²³R²⁴—CR²⁵R²⁶R²⁷, —CR²⁸R²⁹—CR³⁰R³¹—CR³²R³³R³⁴, —CR³⁵R³⁶—CR³⁷R³⁸—CR³⁹R⁴⁰—CR⁴¹R⁴²R⁴³; alkyl groups with 2-25 carbon atoms, which groups are substituted with one or more of the residues R¹² to R⁴³; alkenyl groups with 2-25 carbon atoms, which groups are substituted with one or more of the residues R¹² to R⁴³; alkinyl groups with 2-25 carbon atoms, which groups are substituted with one or more of the residues R¹² to R⁴³; alkoxy groups with 2-25 carbon atoms, which groups are substituted with one or more of the residues R¹² to R⁴³; aryl groups with 2-25 carbon atoms, which groups are substituted with one or more of the residues R¹² to R⁴³; heteroaryl groups with 2-25 carbon atoms, which groups are substituted with one or more of the residues R¹² to R⁴³; heterocyclyl groups with 2-25 carbon atoms, which groups are substituted with one or more of the residues R¹² to R⁴³,
R¹²-R⁴⁷ represent, independently of each other, the following residues:
—H, —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —N₃, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COCN, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CON(CH₃)₂, —CON(C₂H₅)₂, —CON(C₃H₇)₂, —CON(cyclo-C₃H₅)₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—C(═NH)—NH₂, —O—CO—NH₂, —O—CO—NHCH₃, —O—CO—NHC₂H₅, —O—CO—NHC₃H₇, —O—CO—NH-cyclo-C₃H₅, —O—CO—N(CH₃)₂, —O—CO—N(C₂H₅)₂, —O—CO—N(C₃H₇)₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, -Ph, —CH₂-Ph, —CPh₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C≡CH; as well as pharmacologically acceptable salts, solvates, hydrates, complex compounds, enantiomers, diastereomers and racemates of the aforementioned compounds.

The compounds of formula (I) according to the present invention can either be administered per se or in the form of their pharmacologically active salt. Since the compounds of the general formula (I) may have both basic and acidic properties, salts of these compounds can be prepared according to conventional methods.

Suitable examples for salts of compounds according to formula (I) comprise acid addition salts, alkali metal salts, and salts with amines. Alkali metal salts such as sodium salt, potassium salt, lithium salt, or magnesium salt, calcium salt, alkyl amino salts, or amino acid salts, for instance, of basic amino acids such as lysine, can be mentioned. The following acids can be counted among the acids forming an acid addition salt of the compound of formula (I): sulfuric acid, sulfonic acid, phosphoric acid, nitric acid, nitrous acid, perchloric acid, hydrobromic acid, hydrochloric acid, formic acid, acetic acid, propionic acid, succinic acid, oxalic acid, gluconic acid (glyconic acid, dextronic acid), lactic acid, malic acid, tartaric acid, tartronic acid (hydroxymalonic acid, hydroxypropane diacid), fumaric acid, citric acid, ascorbic acid, maleic acid, malonic acid, hydroxymaleic acid, pyruvic acid, phenylacetic acid, (o, m, p)-toluic acid, benzoic acid, p-aminobenzoic acid, p-hydroxybenzoic acid, salicylic acid, p-aminosalicylic acid, methanesulfonic acid, ethanesulfonic acid, hydroxyethanesulfonic acid, ethylenesulfonic acid, p-toluenesulfonic acid, naphthylsulfonic acid, naphthylamine sulfonic acid, sulfanilic acid, camphersulfonic acid, china acid (quininic acid), o-methylmandelic acid, hydrogen-benzenesulfonic acid, picric acid (2,4,6-trinitrophenol), adipic acid, d-o-tolyltartaric acid, amino acids such as methionine, tryptophane, arginine and especially acidic amino acids such as glutamic acid or aspartic acid.

Depending on the kind of compound of the general formula (I), betaine forms are possible, too.

Preferred are compounds of the general formula (I), wherein the chiral center at position 2 of the propionic acid chain has S configuration, as shown in formula (V):

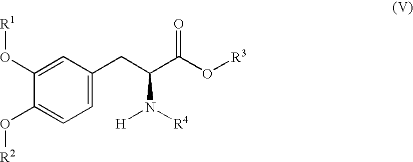

Further preferred are such compounds, in which R¹ and R² represent an acetyl group and an alkyl group. Thus, compounds of the general formula (II) are obtained:

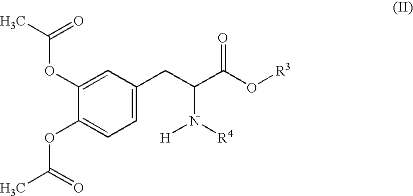

Therein, R³ and R⁴ have the meaning indicated above. Again, the S configuration at carbon atom 2 of the propionic acid chain is preferred.

Furthermore, such compounds are preferred in which either R³ or R⁴ represents hydrogen. If R⁴ represents hydrogen, the following compounds of the general formula (III) are obtained:

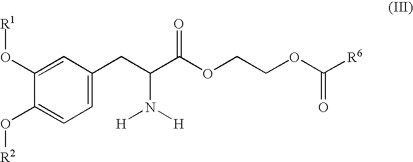

wherein R¹, R² and R⁶ have the meanings indicated above. Here, too, it is preferred that the carbon atom bearing the amino group has S configuration.

In the case that R³ represents hydrogen, compounds of the type (IV) are obtained:

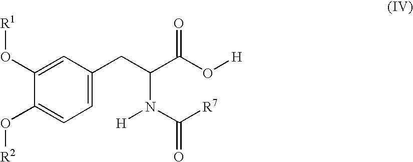

wherein R¹, R² and R⁷ have the meanings indicated above. Again, the S configuration at carbon atom 2 of the propionic acid chain is preferred. Moreover, it is preferred that the groups R¹ and R² in formula (IV) both represent hydrogen or an acetyl group or an alkyl group.

Preferably, the groups —CO—R⁶ and —CO—R⁷ represent fatty acid groups, derived from the corresponding fatty acids HOOC—R⁶ and HOOC—R⁷. In this context, the residues —R⁶ and —R⁷ represent the carbon chain of the fatty acids. Said carbon chain consists of 2-25 and preferably of 5-9 carbon atoms in the case of substituted carbon chains. It is known that said carbon chains of the fatty acids can be saturated or unsaturated, may be branched and in particular, that they may comprise one or more isolated, conjugated, or polyconjugated double and/or triple bonds.

Further preferred are in particular compounds of the general formula:

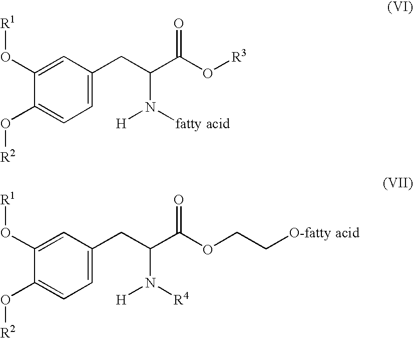

wherein “fatty acid” represents an acyl group, in particular the fatty acid represented herein. Herein, the carbon chains of said fatty acids are also referred to as R⁶ and R⁷.

In the case of a carbon chain substituted with one or more hydroxy groups, alkoxy groups, thio groups, mercapto groups, amino groups, halogen groups, carbonyl groups, carboxyl groups and/or nitro groups or containing a ring system, a number of carbon atoms from 7 to 25 is preferred. In any carbon chain represented by the residues R⁶ and R⁷, a number from 5-24 carbon atoms is preferred, more preferred are 7-23 carbon atoms, still more preferred are 9-22 carbon atoms, still more preferred are 11-21 carbon atoms, and especially preferred are 13-20 carbon atoms.

The lipoic acid residue as well as the dihydrolipoic acid residue are preferred for the cyclic or substituted carbon residues.

Below, the carboxylic acid residues as well as their nomenclature are further described. The following fatty acid is an example for the compounds HOOC—R⁶ and HOOC—R⁷:

H₃C—(CH₂)₇—C≡C—CH₂—CH═C H—(CH₂)₄—COOH

This fatty acid is designated as 6,9-octadecenoic acid or octadec-6-en-9-oic acid. The carboxylic acid residue represented by the residues —CO—R⁶ or —CO—R⁷

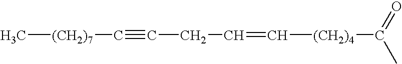

is designated as 6,9-octadecenynoyl or octadec-6-en-9-ynoyl, respectively. The carbon chain of the carboxylic acid represented by the residue group —R⁶ and —R⁷ is as follows:

H₃C—(CH₂)₇—C≡C—CH₂—CH═CH—(CH₂)₄—

and is designated as 5,8-heptadecenynyl or heptadec-5-en-8-ynyl, respectively.

For the designation of the position of the multiple bonds in unsaturated fatty acids, a chemical and a biochemical nomenclature has been established. Accordingly, linoleic acid is designated as, for instance, cis-9, cis-12-octadecadienoic acid (chemical nomenclature) or Δ9,12-octadecadienoic acid or octadecadienoic acid (18:2) or octadecadienoic acid 18:2 (n-6) (biochemical or physiological nomenclature), respectively. In the case of octadecadienoic acid 18:2 (n-6) the number of carbon atoms is represented by n and the integer “6” indicates the position of the last double bond. Consequently, 18:2 (n-6) describes a fatty acid with 18 carbon atoms, two double bonds and a distance of 6 carbon atoms from the last double bond to the terminal methyl group.

Since the inventive compounds either comprise a carboxylic acid residue linked to the carboxylate group of DOPA (2-amino-3-(3,4-dihydroxyphenyl)-propionic acid) via an ester bond with an ethylene glycol group situated inbetween (see formula III) or contain a carboxylic acid residue linked via an amide bond to the amino group of DOPA (see formula IV), carboxylic acids and especially fatty acids which, according to the invention, can be used for the synthesis of the compounds of general formula (I) are listed below. The corresponding carbonyl groups are represented by the residues —CO—R⁶ and —CO—R⁷ and the corresponding carbon chains of the carbonyl acids are represented by the residues —R⁶ and R⁷.

Table 1 shows a list of linear and saturated carboxylic acids.

TABLE 1
Linear Saturated Carboxylic Acids

| Systematic Name | Trivial Name | Numerical Designation |
|---|---|---|
| ethanoic acid | acetic acid | 2:0 |
| propanoic acid | propionic acid | 3:0 |
| butanoic acid | butyric acid | 4:0 |
| hexanoic acid | caproic acid | 6:0 |
| octanoic acid | caprylic acid | 8:0 |
| decanoic acid | capric acid | 10:0 |
| dodecanoic acid | lauric acid | 12:0 |
| tetradecanoic acid | myristic acid | 14:0 |
| hexadecanoic acid | palmitic acid | 16:0 |
| heptadecanoic acid | margaric acid | 17:0 |
| octadecanoic acid | stearic acid | 18:0 |
| eicosanoic acid | arachidic acid | 20:0 |
| docosanoic acid | behenic acid | 22:0 |
| tetracosanoic acid | lignoceric acid | 24:0 |

Table 2 shows a list of monoolefinic fatty acids.

TABLE 2
Monoolefinic Fatty Acids

| Systematic Name | Trivial Name | Numerical Designation |
|---|---|---|
| cis-9-tetradecenoic acid | myristoleic acid | 14:1(n-5) |
| cis-9-hexadecenoic acid | palmitoleic acid | 16:1(n-7) |
| cis-6-octadecenoic acid | petroselinic acid | 18:1(n-12) |
| cis-9-octadecenoic acid | oleic acid | 18:1(n-9) |
| cis-11-octadecenoic acid | vaccenic acid | 18:1(n-7) |
| cis-9-eicosenoic acid | gadoleic acid | 20:1(n-11) |
| cis-11-eicosenoic acid | gondoic acid | 20:1(n-9) |
| cis-13-docosenoic acid | erucic acid | 22:1(n-9) |
| cis-15-tetracosenoic acid | nervonic acid | 24:1(n-9) |
| t9-octadecenoic acid | elaidic acid | |
| t11-octadecenoic acid | t-vaccenic acid | |
| t3-hexadecenoic acid | | trans-16:1 n-13 |

Table 3 shows a list of polyunsaturated fatty acids.

TABLE 3
Polyunsaturated Fatty Acids

| | | Numerical |
|---|---|---|
| Systematic Name | Trivial Name | Designation |
| 9,12-octadecadienoic acid | linoleic acid | 18:2(n-6) |
| 6,9,12-octadecatrienoic acid | γ-linoleic acid | 18:3(n-6) |
| 8,11,14-eicosatrienoic acid | dihomo-γ-linolenic acid | 20:3(n-6) |
| 5,8,11,14-eicosatetraenoic acid | arachidonic acid | 20:4(n-6) |
| 7,10,13,16-docosatetraenoic acid | — | 22:4(n-6) |
| 4,7,10,13,16-docosapentaenoic acid | — | 22:5(n-6) |
| 9,12,15-octadecatrienoic acid | α-linoleic acid | 18:3(n-3) |
| 6,9,12,15-octadecatetraenoic acid | stearidonic acid | 18:4(n-3) |
| 8,11,14,17-eicosatetraenoic acid | — | 20:4(n-3) |
| 5,8,11,14,17-eicosapentaenoic acid | EPA | 20:5(n-3) |
| 7,10,13,16,19-docosapentaenoic acid | DPA | 22:5(n-3) |
| 4,7,10,13,16,19-docosahexaenoic acid | DHA | 22:6(n-3) |
| 5,8,11-eicosatrienoic acid | mead acid | 20:3(n-9) |
| 9c 11t 13t eleostearic acid | | |
| 8t 10t 12c calendic acid | | |
| 9c 11t 13c catalpic acid | | |
| 4, 7, 9, 11, 13, 16, 19 | stellaheptaenoic acid | |
| docosaheptadecanoic acid | | |
| | taxoleic acid | all-cis-5,9; 18:2 |
| | pinolenic acid | all-cis-5,9,12; 18:3 |
| | sciadonic acid | all-cis-5,11,14; 20:3 |

Table 4 shows a list of acetylenic fatty acids.

TABLE 4
Acetylenic Fatty Acids

| Systematic Name | Trivial Name |
|---|---|
| 6-octadecynoic acid | tariric acid |
| t11-octadecen-9-ynoic acid | santalbic or ximenynic acid |
| 9-octadecynoic acid | stearolic acid |
| 6-octadecen-9-ynoic acid | 6,9-octadecenynoic acid |
| t10-heptadecen-8-ynoic acid | pyrulic acid |
| 9-octadecen-12-ynoic acid | crepenynic acid |
| t7,t11-octadecadiene-9-ynoic acid | heisteric acid |
| t8,t10-octadecadiene-12-ynoic acid | — |
| 5,8,11,14-eicosatetraynoic acid | ETYA |

Preferably, the following carboxylic acids are used for the synthesis of the inventive compounds: linoleic acid, γ-linolenic acid, dihomo-γ-linolenic acid, arachidonic acid, 7,10,13,16-docosatetraenoic acid, 4,7,10,13,16-docosapentaenoic acid, α-linolenic acid, stearidonic acid, 8,11,14,17-eicosatetraenoic acid, EPA, DPA, DHA, mead acid, eleostearic acid, calendic acid, catalpic acid, stellaheptaenoic acid, taxoleic acid, pinolenic acid, sciadonic acid, retinoic acid, isopalmitic acid, pristanic acid, phytanic acid, 11,12-methyleneoctadecanoic acid, 9,10-methylenhexadecanoic acid, coronaric acid, (R,S)-lipoic acid, (S)-lipoic acid, (R)-lipoic acid, 6,8-bis(methylsulfanyl)-octanoic acid, 4,6-bis(methylsulfanyl)-hexanoic acid, 2,4-bis(methylsulfanyl)-butanoic acid, 1,2-dithiolane carboxylic acid, (R,S)-6,8-dithiane octanoic acid, (R)-6,8-dithiane octanoic acid, (S)-6,8-dithiane octanoic acid, tariric acid, santalbic acid, stearolic acid, 6,9-octadecenynoic acid, pyrulic acid, crepenynic acid, heisteric acid, t8,t10-octadecadiene-12-inoic acid, ETYA, cerebronic acid, hydroxynervonic acid, ricinoleic acid, lesquerolic acid, brassylic acid and thapsic acid.

Among the carboxylic acids γ-linolenic acid, α-linolenic acid, EPA, DHA, (R,S)-lipoic acid, (S)-lipoic acid and (R)-lipoic acid, 6,8-bis(methylsulfanyl)-octanoic acid, 4,6-bis(methylsulfanyl)-hexanoic acid, 2,4-bis(methylsulfanyl)-butanoic acid, 1,2-dithiolane carboxylic acid are particularly preferred.

Dodecanoyl, hexadecanoyl, octadecanoyl, eicosanoyl, docosanoyl, tetracosanoyl, cis-9-tetradecenoyl, cis-9-hexadecenoyl, cis-6-octadecenoyl, cis-9-octadecenoyl, cis-11-octadecenoyl, cis-9-eicosenoyl, cis-11-eicosenoyl, cis-13-docosenoyl, cis-15-tetracosenoyl, 9,12-octadecadienoyl, 6,9,12-octadecatrienoyl, 8,11,14-eicosatrienoyl, 5,8,11,14-eicosatetraenoyl, 7,10,13,16-docosatetraenoyl, 4,7,10,13,16-docosapentaenoyl, 9,12,15-octadecatrienoyl, 6,9,12,15-octadecatetraenoyl, 8,11,14,17-eicosatetraenoyl, 5,8,11,14,17-eicosapentaenoyl, 7,10,13,16,19-docosapentaenoyl, 4,7,10,13,16,19-docosahexaenoyl, 5,8,11-eicosatrienoyl, 1,2-dithiolan-3-pentanoyl, 6,8-dithianoctanoyl, docosaheptadecanoyl, eleostearoyl, calendoyl, catalpoyl, taxoleoyl, pinolenoyl, sciadonoyl, retinoyl, 14-methylpentadecanoyl, pristanoyl, phytanoyl, 11,12-methyleneoctadecanoyl, 9,10-methylenehexadecanoyl, 9,10-epoxystearoyl, 9,10-epoxyoctadec-12-enoyl, 6-octadecynoyl, t11-octadecen-9-ynoyl, 9-octadecynoyl, 6-octadecen-9-ynoyl, t10-heptadecen-8-ynoyl, 9-octadecen-12-ynoyl, t7,t11-octadecadiene-9-ynoyl, t8,t10-octadecadiene-12-ynoyl, 5,8,11,14-eicosatetraynoyl, 2-hydroxytetracosanoyl, 2-hydroxy-15-tetracosenoyl, 12-hydroxy-9-octadecenoyl and 14-hydroxy-11-eicosenoyl are preferred as residues —CO—R⁶ and —CO—R⁷. Moreover, the aforementioned fatty acid residues may also be substituted with one, two or more substituents selected from the group referred to as R¹²-R⁴⁷.

The following groups are particularly preferred as residues —CO—R⁶ and —CO—R⁷: 9,12-octadecadienoyl, 6,9,12-octadecatrienoyl, 8,11,14-eicosatrienoyl, 5,8,11,14-eicosatetraenoyl, 9,12,15-octadecatrienoyl, 6,9,12,15-octadecatetraenoyl, 8,11,14,17-eicosatetraenoyl, 5,8,11,14,17-eicosapentaenoyl, 7,10,13,16,19-docosapentaenoyl, 4,7,10,13,16,19-docosahexaenoyl, 5,8,11-eicosatrienoyl, 1,2-dithiolane-3-pentanoyl and 6,8-dithianeoctanoyl.

The inventive compounds are obtained by protecting or derivatizing both hydroxy groups of L-DOPA and by subsequently forming the amide bond with the fatty acid or respectively carboxylic acid by means of anhydrides or by protecting the amino group of L-DOPA and by forming the ester bond according to known procedures, for instance with an activated carboxylic acid (carboxylic acid chloride, carboxylic acid bromide, carboxylic acid azide, anhydride, carboxylic acid succinimidyl ester and the like). Thereafter, the amino group can be deprotected and may be reacted with the same or a different fatty acid or carboxylic acid, respectively, under formation of an amide bond. Subsequently, the hydroxy protecting groups can be removed.

Furthermore, the present invention relates to pharmaceutical compositions which were manufactured using at least one inventive compound or a salt thereof.

In addition to at least one compound of the general formula (I), the pharmaceutical compositions comprise a pharmacologically acceptable carrier, adjuvant and/or diluents.

The pharmaceutical compositions can be manufactured and administered in form of drops, mouth spray, nose spray, pills, tablets, film coated tablets, multi-layered tablets, suppositories, gels, ointments, syrups, inhalation powders, granulates, emulsions, dispersions, microcapsules, capsules, powders or solutions for injection. Additionally, the inventive pharmaceutical compositions comprise formulations such as multi-layered tablets for controlled and/or continuous release of the active agent as well as micro-encapsulated formulations as a specific dosage form.

Such formulations are also suitable for inhalation or for intravenous, intraperitoneal, intramuscular, subcutaneous, mucocutaneous, oral, rectal, transdermal, topical, buccal, intradermal, intragastric, intracutaneous, intranasal, intrabuccal, percutaneous or sublingual administration.

For example, lactose, starch, sorbitol, sucrose, cellulose, magnesium stearate, dicalcium phosphate, calcium sulfate, talc, mannitol, ethyl alcohol and the like can be used as pharmacologically acceptable carriers. Powders and tablets may consist of such a carrier to an extent of 5 to 95%.

Furthermore, starch, gelatine, natural sugars and both natural and synthetic gums such as acacia gum or guar gum, sodium alginate, carboxymethylcellulose, polyethylene glycol and waxes can be used as binders. Boric acid, sodium benzoate, sodium acetate, sodium chloride and the like can be used as lubricants.

Additionally, disintegrants, coloring agents, flavoring agents and/or binders may be added to the pharmaceutical compositions.

Liquid formulations comprise solutions, suspensions, sprays and emulsions, such as injection solutions on the basis of water or on the basis of water-propylene glycol for parenteral injection.

Preferably, low-melting waxes, fatty acid esters, and glycerides are used for the preparation of suppositories.

Capsules are prepared from, for instance, methylcellulose, polyvinyl alcohol or denaturated gelatine or starch.

Starch, sodium carboxymethyl starch, natural and synthetic gums such as locust bean gum, karaya gum, guar, tragacanth and agar as well as cellulose derivatives such as methylcellulose, sodium carboxymethylcellulose, microcrystalline cellulose and alginates, clays and bentonite can be used as disintegrants. Said components can be used in quantities of 2 to 30% by weight.

Sugars, starches from corn, rice or potatoes, natural gums such as acacia gum, gelatine, tragacanth, alginic acid, sodium alginate, ammonium calcium alginate, methylcellulose, sodium carboxymethylcellulose, hydroxypropyl methylcellulose, polyvinylpyrrolidone and inorganic compounds such as magnesium aluminum silicates can be added as binders. The binders can be added in quantities of 1 to 30% by weight.

Stearates such as magnesium stearate, calcium stearate, potassium stearate, stearic acid, high-melting waxes as well as water-soluble lubricants such as sodium chloride, sodium benzoate, sodium acetate, sodium oleate, polyethylene glycol and amino acids such as leucine can be used as lubricants. Such lubricants can be used in quantities of 0.05 to 15% by weight.

The compounds according to the invention and the pharmaceutical compositions described above are used, for instance for the treatment and/or the prophylaxis of, or respectively for the manufacture of a pharmaceutical formulation for the treatment and/or the prophylaxis of movement disorders, in particular movement disorders such as early-onset drug-induced dyskinesias, akathisia, parkinsonian features and in particular rigidity and tremor, further extrapyramidal disorders such as segmented and generalized dystonias, drug-induced extrapyramidal symptoms, movement disorders due to other causes than Parkinson's disease as well as different forms of parkinsonian syndromes (endogenous, atherosclerotic, postencephalitic, drug-induced), neurodegenerative diseases, Alzheimer's disease, Parkinson's disease, hemiparkinson-hemiatrophy, Parkinson's syndrome due to or associated with hydrocephalus (hydrocephaly), oxygen deficiency, infections of the brain (encephalitis), manganese poisoning, carbon monoxide (CO) poisoning, 1-methyl-4-phenyl-1,2,3,6-tetrahydropyridine (MPTP) poisoning and cyanide poisoning, parathyroid diseases, brain tumor, brain lesions, infarctions, Lewy body disease, frontotemporal dementia, Lytico-Bodig disease (parkinsonism/dementia/amyotrophic lateral sclerosis), striatonigral degeneration, Shy-Drager syndrome, sporadic olivopontocerebellar degeneration, progressive atrophy of the globus pallidus, progressive supranuclear palsy, Hallervorden-Spatz disease, Huntington's disease, X-linked dystonia-parkinsonism (Lubag), mitochondrial cytopathy with striatal necrosis, neuroacanthocytosis, restless legs syndrome (refers to dysesthesias and paresthesias with origin at the ankle joint which may spread across the lower leg and knee and into the thigh or persist on one level; in a few cases arms and hands are also involved) and Wilson's disease.

The term movement disorders refers in particular to spastic disorders, hyperkinesias, dystonias, athetoses, dyskinesias, myoclonus syndrome, Wilson's disease, choreatic syndromes, tics, Tourette's disorder, ballism, tremor syndromes and Parkinson's disease.

Another aspect of the present invention relates to pharmaceutical compositions containing, in addition to the at least one inventive compound, one, two or more additional pharmacologically active agents suitable for the treatment and/or prophylaxis of movement disorders, neurodegenerative diseases, Alzheimer's disease, Parkinson's disease, hemiparkinson-hemiatrophy, Parkinson's syndrome, rigidity, tremor, dystonias, Lewy body disease, frontotemporal dementia, Lytico-Bodig disease (parkinsonism/dementia/amyotrophic lateral sclerosis), striatonigral degeneration, Shy-Drager syndrome, sporadic olivopontocerebellar degeneration, progressive atrophy of the globus pallidus, progressive supranuclear palsy, Hallervorden-Spatz disease, Huntington's disease, X-linked dystonia-parkinsonism (Lubag), mitochondrial cytopathy with striatal necrosis, neuroacanthocytosis, restless legs syndrome, Wilson's disease.

Amongst others, dopamine receptor agonists such as bromocriptine, cabergoline, lisuride, dihydroergocriptine, dopamine agonists, entacapone, pramipexol, pergolide mesylate, pergolide, ropinirole, NMDA glutamate receptor antagonists such as amantadine and budipine, monoamine oxidase B inhibitors such as selegiline, catechol-O-methyltransferase inhibitors such as entacapone, anticholinergics such as benzatropine, biperiden, bornaprine, procyclidine, trihexyphenidyl, antioxidants such as vitamin C and vitamin E are counted among said further active agents.

### DESCRIPTION OF FIGURES

FIG. 1A shows the microdialysis in the nucleus accumbens of a freely moving rat. The arrow indicates the moment of injection (ip: intraperitoneal) of L-DOPA (25 mg/kg body weight) 30 minutes after the administration of benserazide (10 mg/kg body weight, ip).

FIG. 1B shows the microdialysis in the nucleus accumbens of a freely moving rat. The arrow indicates the moment of injection (ip: intraperitoneal) of L-DOPA (50 mg/kg body weight) 30 minutes after the administration of benserazide (10 mg/kg body weight, ip, in 0.9% NaCl).

FIG. 2 shows the microdialysis in the nucleus accumbens of a freely moving rat (control experiment). The arrow indicates the moment of injection (ip: intraperitoneal) of solutol 30 minutes after the administration of benserazide (10 mg/kg body weight, ip, in 0.9% NaCl).

FIG. 3 shows the microdialysis in the nucleus accumbens of a freely moving rat. The arrow indicates the moment of injection (ip: intraperitoneal) of diacetyl-DOPA-ethylene glycol α-lipoic acid (equimolar dose to 25 mg/kg body weight L-DOPA) 30 minutes after the administration of benserazide (10 mg/kg body weight, ip in 0.9% NaCl).

FIG. 4 shows the microdialysis in the nucleus accumbens of a freely moving rat. The arrow indicates the moment of injection (ip: intraperitoneal) of diacetyl-DOPA-ethylene glycol α-lipoic acid (equimolar dose to 50 mg/kg body weight L-DOPA) 30 minutes after the administration of benserazide (10 mg/kg body weight, ip).

### EXAMPLES

### Example 1

### Synthesis of O,O′-diacetyl-L-DOPA-ethylene glycol lipoic acid ester (diacetyl-DOPA-ethylene glycol lipoic acid ester; compound 1)

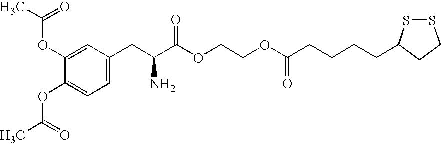

Lipoic acid was converted to the lipoic acid monoethylene glycol ester by means of an excess of ethylene glycol and DCC. L-DOPA was converted to Fmoc-L-DOPA by Fmoc-succinimide and was acetylated under Schotten-Baumann conditions to N-Fmoc-O,O′-diacetyl-L-DOPA by acetic acid anhydride. By the conversion of the above lipoic acid monoethylene glycol ester by means of DDC, the coupling product N-Fmoc-O,O′-diacetyl-L-DOPA ethylene glycol-rac-lipoic acid ester was obtained. The Fmoc protecting group was cleaved by tetrabutylammonium fluoride in DMF.

Purity (HPLC) 81-85%, clear yellow oil

¹³C-NMR (100.6 MHz, d₄-methanol), δ (ppm):

20.47; 25.71; 29.74; 34.76; 35.68; 39.33; 41.07; 41.27; 57.51; 61.05; 66.47; 66.85; 121.66; 121.82; 129.52; 145.10; 146.18; 167.40; 167.63; 175.36; 176.31.

### Example 2

### Synthesis of O,O′-diacetyl-L-DOPA-(R,S)-lipoic acid amide (diacetyl-DOPA-lipoic acid amide; compound 2)

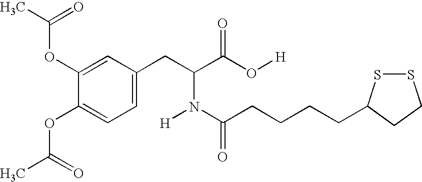

O,O′-diacetyl-L-DOPA-rac-lipoic acid amide was obtained by reacting L-DOPA-rac-lipoic acid amide with acetic acid anhydride under mildly basic reaction conditions.

Purity (HPLC)>95%, clear yellow oil

¹³C-NMR (100.6, CDCl₃), δ (ppm):

20.65; 25.16; 28.67; 34.50; 35.99; 36.48; 38.43; 40.17; 52.74; 56.29; 123.43; 124.59; 127.39; 134.70; 141.05; 141.84; 168.33; 168.39; 173.46; 173.67.

### Example 3

### Synthesis of L-DOPA-(D,L)-lipoic acid amide (DOPA-lipoic acid amide; compound 3)

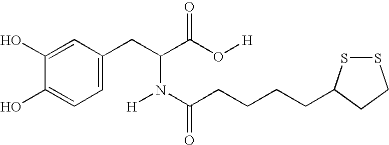

L-DOPA-rac-lipoic acid amide was obtained by the N-acylation of L-Dopa with activated lipoic acid derivatives such as lipoic acid chloride or lipoic acid succinimidyl ester under mildly basic conditions. Lipoic acid chloride was obtained from lipoic acid and oxalyl chloride, lipoic acid succinimidyl ester was obtained from lipoic acid, hydroxysuccinimide and DCC.

Purity (HPLC) 97%, yellow, highly viscous oil

¹³C-NMR (100.6, d₄-methanol), δ (ppm):

26.58; 29.60; 35.69; 36.60; 37.83; 39.31; 46.23; 55.00; 57.43; 116.24; 117.23; 121.60; 129.85; 145.13; 146.16; 174.99; 175.84.

### Example 4

### Synthesis of L-DOPA-tri-(D,L)-lipoic acid derivative (DOPA-tri-lipoic acid derivative; compound 4)

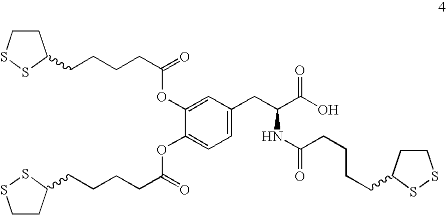

Attempts were made to obtain the target compound 4 by direct acylation of L-Dopa with an excess of the acylation means lipoic acid hydroxysuccinimidyl ester.

Reaction Mixture:

| Quantity | | Compound | Molar Weight | mmol |
|---|---|---|---|---|
| 0.80 | g | L-DOPA | 197.19 | 4.06 |
| 5.55 | g | lipoic acid hydroxysuccinimidyl ester | 303.39 | 16.00 |
| 3.5 | ml | triethylamine | 101.19 | 25.00 |

### Realization

Reaction under argon and under the exclusion of light.

4.85 g of lipoic acid hydroxysuccinimidyl ester were dissolved in 35 ml of ethyl acetate and 20 ml of acetone nitrile. The solution was degassed under vacuum and deaerated with argon. 0.80 g of L-DOPA were dissolved in 20 ml of water and 3.5 ml of triethylamine were added. The solution was degassed again under vacuum and deaerated with argon. The solution was stirred overnight at room temperature. Additionally, 0.7 g of lipoic acid hydroxysuccinimidyl ester were added and stirring was continued for another 3.5 hours at room temperature. The reaction mixture was added to a vigorously stirred mixture consisting of 150 ml of ethyl acetate and 50 ml of water. This mixture was carefully acidified using diluted hydrochloric acid. The phases were separated and the organic phase was washed twice with saturated NaCl solution and dried over sodium sulfate. The solution was concentrated on the rotary evaporator. The residue was chromatographed on 300 ml of silica gel (eluent: methylene chloride/ethyl acetate/formic acid=8:2:0.075 to 5:5:0.075)

### TLC Conditions

Solvent: CH₂Cl₂/ethyl acetate/HCO₂H=5:5:0.075; detection: UV; I₂ chamber or respectively KMnO₄ solution

R_{f}=0.29

### HPLC—Scatter Detector

Rₜ=16.86 min (81.5%)

In fact, compound 4 could be obtained by acylation with lipoic acid hydroxysuccinimidyl ester. A considerably more effective approach for obtaining the target compound consists in two-step synthesis. Thereby, 1.8 g of the target compound 4 (yield: 39%) could be obtained.

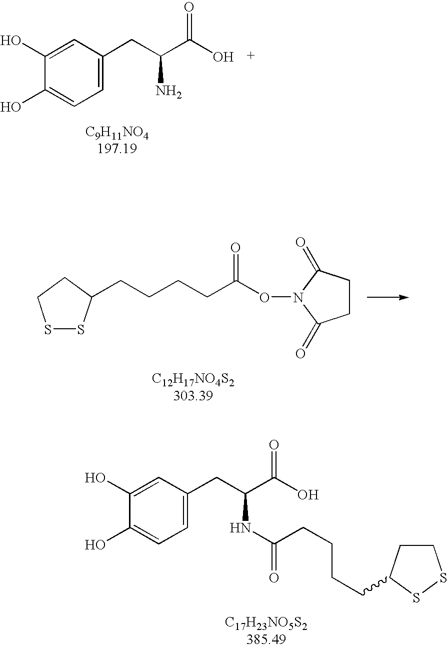

Reaction Mixture (under the exclusion of light):

| Quantity | Compound | Molar Weight | mmol |
|---|---|---|---|
| 2.25 g | L-DOPA | 197.19 | 11.41 |
| 1.86 g | lipoic acid hydroxysuccinimidyl ester | 303.39 | 6.13 |
| 4.00 ml | triethylamine | | |

### Realization

2.25 g of L-DOPA were suspended in 50 ml of water and 50 ml of acetone nitrile. The suspension was degassed under vacuum and deaerated with argon. 4.0 ml of triethylamine were added. After 10 minutes, a solution of 1.86 g of lipoic acid hydroxysuccinimidyl ester in 60 ml of ethyl acetate was quickly added under vigorous stirring. After 30 minutes, 60 ml of water and 100 ml of ethyl acetate were added. For acidification, diluted hydrochloric acid was added under vigorous stirring. The aqueous phase was separated and the organic phase was washed twice with saturated NaCl solution and dried over sodium sulfate. The solution was concentrated on the rotary evaporator (bath temperature: 32° C.) to a residue of 18.36 g. 0.705 g of said solution were completely concentrated which led to a yield of 83 mg of a residue (2.08 g of crude product). The solution was divided into two equal portions and further reacted as described below:

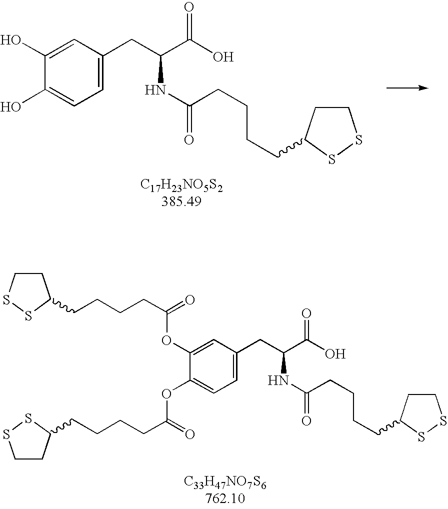

I. Reaction Mixture (under the exclusion of light):

| Quantity | | Compound | Molar Weight | mmol |
|---|---|---|---|---|
| 8.8 | g | solution crude product | 385.49 | 2.7 |
| 1.47 | g | lipoic acid | 206.32 | 7.1 |
| 1.20 | g | DCC | 206.33 | 5.8 |

1.24 g of lipoic acid were dissolved in 40 ml of dry methylene chloride. 1.20 g of DCC dissolved in 10 ml of methylene chloride were added. After 30 seconds, the L-DOPA solution +5 ml of methylene chloride were added. After 5 minutes, a spatula tip of DMAP was added. After 2 hrs, a TLC sample contained only traces of the desired product. 1.3 ml of triethylamine were added and stirred overnight at room temperature. 20 ml of citric acid solution and 10 ml of diluted hydrochloric acid were added and the reaction was stirred vigorously for 1 h. After the addition of 120 ml of ethyl acetate and 50 ml of water the phases were separated. The organic phase was first washed with saturated citric acid solution and subsequently with saturated NaCl solution.

### TLC Conditions

Solvent: CH₂Cl₂/ethyl acetate/HCO₂H=5:5:0.075; detection: UV; or respectively KMnO₄ solution

R_{f}=0.78; 0.67; 0.63; 0.54 impurities

R_{f}=0.26 product

II. Reaction Mixture (under the exclusion of light):

| Quantity | | Compound | Molar Weight | mmol |
|---|---|---|---|---|
| 8.8 | g | solution crude product | 385.49 | 2.7 |
| 1.47 | g | lipoic acid | 206.32 | 7.1 |
| 1.20 | g | DCC | 206.33 | 5.8 |
| 1.3 | ml | triethylamine | 101.19 | 9.45 |

As described under I; however, 10 ml of methylene chloride and 1.3 ml of triethylamine were added to the solution of the crude product (8.8 g). After 3 hrs, a TLC sample contained no reactant; subsequently, 120 ml of ethyl acetate and 40 ml of citric acid solution were added. The mixture was stirred vigorously overnight. Additionally, 20 ml of diluted hydrochloric acid were added and the phases were separated. The organic phase was first washed with saturated citric acid solution and subsequently with saturated NaCl solution.

### TLC Conditions

Solvent: CH₂Cl₂/ethyl acetate/HCO₂H=5:5:0.075; detection: UV; or respectively KMnO₄ solution

R_{f}=0.65 impurity

R_{f}=0.26 product

According to TLC, the reaction mixtures I and II seemed to contain approximately the same quantity of the product; subsequently, they were combined, concentrated under vacuum and the residue was chromatographed on 300 ml of silica gel (eluent: methylene chloride/ethyl acetate/formic acid=8:2:0.075 to 5:5:0.075).

5 g of the product fraction (246.34 g) were extracted and completely concentrated on the rotary evaporator. 38 mg of a polymer residue were obtained. Product in the remaining solution: 1.83 g (39% with respect to the lipoic acid hydroxysuccinimidyl ester used).

### HPLC—Scatter Detector

Rₜ=18.48 min (93.5%)

NMR results for compound 4:

¹H-NMR (400.13 MHz, DMSO-D6):

δ [ppm]=1.22 m (2H); 1.41 m (6H); 1.59 m (10H); 1.84 m (3H); 2.03 t (2H); 2.38 m (3H); m (2H); 2.51 m (6H); 2.82 m (1H); 3.10 m (7H); 3.51 m (1H); 3.58 m (2H); 4.42 m (1H); 6.86-7.12 m (3H)

¹³C-NMR (100.625 MHz, CD₃OD):

δ [ppm]=24.30; 24.33; 24.57; 25.08; 28.20; 28.27; 33.21; 33.43; 34.21; 35.06; 35.11; 36.02; 38.23; 38.27; 39.99; 40.08; 53.05; 56.20; 123.25; 124.12; 126.95; 127.23; 136.66; 140.56; 141.60; 163.05; 170.61; 170.68; 172.39; 173.04

### Example 5

### Synthesis of 3-benzene[1,3]dioxol-5-yl-2-(5-[1,2]dithiolane-3-yl-pentanoylamino)-propionic acid ethyl ester (compound 5)

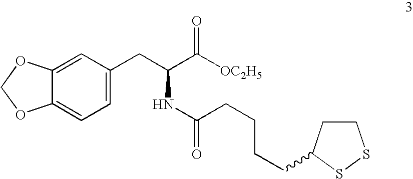

The synthesis of target compound 5 was performed according to the following 5-step reaction scheme:

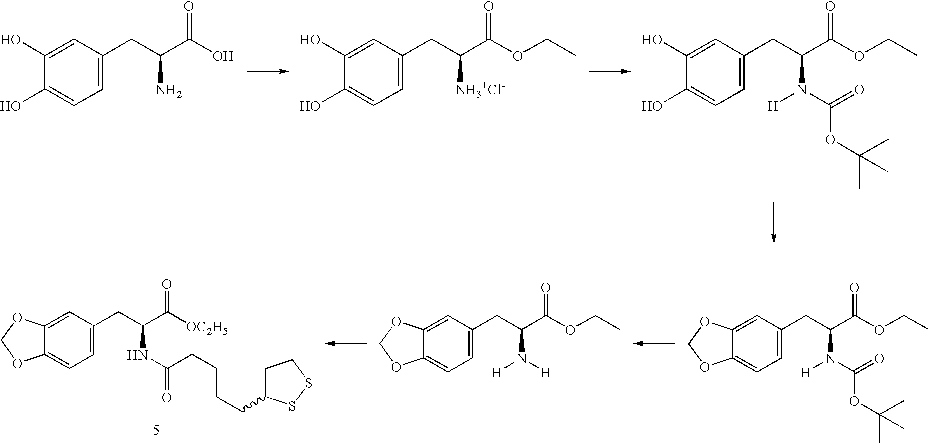

### Description of the Reaction:

2.1 g of L-DOPA (10.6 mmol) were suspended in 90 ml of ethanol. 2.0 ml of thionyl chloride were added dropwise; during this process, L-DOPA was dissolved (formation of HCl). The mixture was heated for 2 hrs under reflux. The volatile components were removed (1. rotary evaporator; 2. high vacuum). 50 ml of saturated sodium hydrogen carbonate solution, 50 ml of acetone nitrile, 3.0 g of sodium hydrogen carbonate and 2.54 g (11.64 mmol) of Boc₂O were added to the amorphous residue (L-DOPA-ethyl ester hydrochloride). The mixture was stirred for 30 minutes at room temperature and for 1.5 hrs at 50-55° C. (water bath) (formation of CO₂). After said period of time, the formation of CO₂ was terminated. Citric acid solution was used for acidification; extraction was performed by means of ethyl acetate, the organic phase was washed with saturated NaCl solution, dried over sodium sulfate and concentrated on the rotary evaporator. The crude product (N-Boc-L-Dopa-ethyl ester) was dissolved under argon in 30 ml of DMF. 3.21 g (12 mmol) of diiodomethane and 6.52 g (25 mmol) of cesium carbonate were added. The mixture was heated for 3 days to 80° C., subsequently the mixture was extracted by shaking with water and methyl t-butyl ether. The organic phase was washed twice with water and once with saturated NaCl solution, dried over sodium sulfate and concentrated on the rotary evaporator. The residue was chromatographed on 450 ml of silica gel 60 (eluent: hexane:ethyl acetate=20:1 to 3:1). 0.82 g (23% with respect to the L-Dopa used) of the methylene-bridged compound were obtained in form of a colorless oil. Said oil was dissolved in 40 ml of ethanol and subsequent to the addition of 10 ml of 6-molar hydrochloric acid (reaction control by means of TLC) the solution was heated to a temperature of 50-55° C. for one hour. The volatile components were removed on the rotary evaporator. The residue was extracted by shaking with saturated sodium hydrogen carbonate solution and ethyl acetate. The organic phase was washed with saturated NaCl solution, dried over sodium sulfate and concentrated on the rotary evaporator to a volume of approx. 40 ml. The remaining solution was degassed under the exclusion of light and mixed with 0.91 g (3 mmol) of lipoic acid hydroxysuccinimidyl ester and 1.5 ml of triethylamine. After 4 hours of stirring at room temperature, the solution was largely (not completely) concentrated on the rotary evaporator and the residue was chromatographed on 150 ml of silica gel 60 (eluent: methylene chloride:ethyl acetate=5:0 to 5:1). 80 g of product fraction were obtained. 0.8 g of the solution were concentrated under vacuum; the residue obtained consisted of 6 mg of a colorless oil which resified soon. This led to a yield of 600 mg (13% with respect to the L-DOPA used). For NMR analyses, 10 ml of the solution were mixed with 0.6 ml of d6-DMSO and the more volatile components were removed on the rotary evaporator.

¹H-NMR (400.13 MHz, DMSO-D6):

δ [ppm]=1.15 t (3H); 1.23 m (2H); 1.56 m (4H); 1.72 m (1H); 1.81 m (1H); 2.05 t (2H); 2.51 m (2H); 2.82 m (1H); 3.51 m (1H); 3.58 m (2H); 4.09 t (2H); 4.43 m (1H); 5.98 s (2H); 6.86-7.12 m (3H)

¹³C-NMR (100.625 MHz, DMSO-D6):

δ [ppm]=13.98; 24.34; 25.09; 28.25; 33.42; 35.07; 36.06; 38.24; 53.02; 56.20; 60.8; 101.13; 112.56; 115.87; 123.45; 133.34; 146; 10; 149.04; 170.62; 172.40

### Example 6

### Synthesis of O,O′-dipropionyl-L-DOPA-oleic acid amide (compound 6)

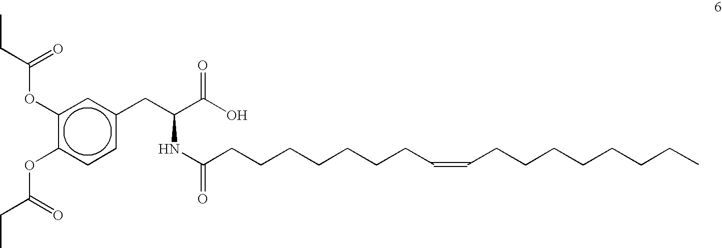

At first, oleic acid was converted to the corresponding acid chloride by means of oxalyl chloride. By the reaction with L-Dopa a moderate yield (23%) of the N-acetylated derivative was obtained after chromatographic purification. By the further reaction with propionic acid anhydride compound 6 was obtained (yield: 93%).

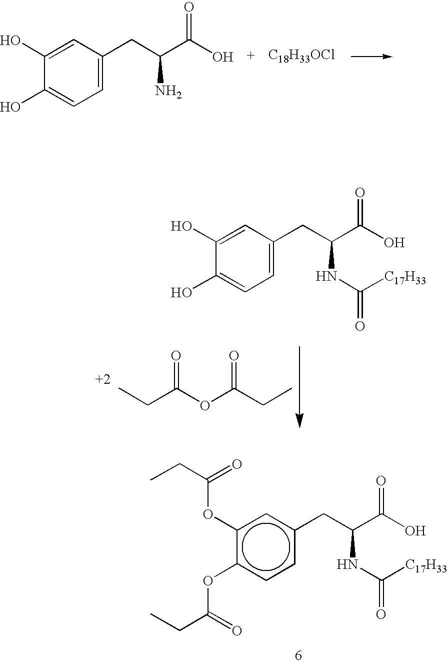

Reaction Mixture:

| Quantity | | Compound | Molar Weight | mmol |
|---|---|---|---|---|
| 1.43 | g | oleic acid (98%) | 285.5 | 5.00 |
| 0.635 | g | oxalyl chloride (d = 1.5) | 126.93 | 5.00 |
| 1.00 | g | L-Dopa | 197.19 | |
| 2 | ml | triethylamine (d = 0.727) | 101.19 | 15.00 |

### Realization

1.43 g of oleic acid were dissolved in 25 ml of methylene chloride and mixed with 0.423 ml of oxalyl chloride. After the addition of 2 drops of DMF, active formation of HCl could be observed. The solution was stirred at room temperature for 2.5 hrs. The volatile components were concentrated on the rotary evaporator (bath temperature: room temperature). The residue was dissolved in 20 ml of ethyl acetate and slowly added dropwise, under argon and while cooling in the ice bath, to a solution of 1.00 g of L-Dopa in 25 ml of water, 30 ml of acetone nitrile and 2 ml of triethylamine. The mixture was stirred for 30 minutes while cooled in the ice bath and for one hour at room temperature. 100 ml of ethyl acetate were added. The mixture was acidified with diluted hydrochloric acid. The phases were separated and the organic phase was washed twice with saturated NaCl solution, dried over sodium sulfate and concentrated on the rotary evaporator. The residue (2.145 g) was chromatographed on 220 ml of silica gel (eluent: 1. 400 ml of methylene chloride/ethyl acetate/acetic acid=5:5:0.2; 2. 500 ml of methylene chloride/ethyl acetate/formic acid=5:5:0.075). 0.53 g (23%) of a colorless, highly viscous oil were obtained. The product (crude product 6a) was directly subjected to further reaction.

### TLC Conditions

Solvent: CH₂Cl₂/ethyl acetate/HCO₂H=5:5:0.075; detection: UV; 12 or respectively KMnO₄ solution

R_{f}=0.69; 0.40; 0.36 impurities

R_{f}=0.25 product

Reaction mixture:

| Quantity | | Compound | Molar Weight | mmol |
|---|---|---|---|---|
| 530 | mg | crude product 6a | 461.64 | 1.148 |
| 850 | mg | propionic acid anhydride | 130.14 | 6.531 |
| 1.5 | ml | triethylamine | 101.19 | 10.78 |

### Realization

530 mg of crude product 6a were dissolved in 10 ml of acetone nitrile and 10 ml of ethyl acetate and mixed with 10 ml of water. The solution was degassed under vacuum and deaerated with argon. During cooling in the ice bath, 1.5 ml of triethylamine were added. Subsequently, a solution of 850 mg of propionic acid anhydride in 7 ml of ethyl acetate was added dropwise. The mixture was stirred for 1 hr at 0° C. and overnight at room temperature. 100 ml of ethyl acetate were added and under strong stirring the mixture was cautiously acidified with diluted hydrochloric acid. The phases were separated and the organic phase was washed with saturated NaCl solution, dried over sodium sulfate and concentrated on the rotary evaporator. The volatile components in the residue were removed during a period of 2 days under high vacuum. A residue of 610 mg (93%) of a colorless, wax-like substance was obtained.

### TLC Conditions

Solvent: CH₂Cl₂/ethyl acetate/HCO₂H=5:5:0.075; detection: I₂-chamber or respectively KMnO₄ solution

R_{f}=0.33

### HPLC—Scatter Detector

Rₜ=27.15 min (95.9%)

NMR analysis regarding compound 6:

¹H-NMR (400.13 MHz, CD₃OD):

δ [ppm]=0.87 t (3H); 1.26 m (26H); 1.56 m (2H); 2.00 m (4H); 2.18 t (2H); 2.53 m (4H); 3.15 m (2H); 4.87 m (1H); 5.33 m (2H); 6.27 m (1H); 6.86-7.12 m (3H); 10.26 s (1H)

¹³C-NMR (100.625 MHz, CD₃OD):

δ [ppm]=9.00; 13.99; 22.58; 25.49; 27.15; 27.38; 29.10; 29.17; 29.24; 29.44; 29.69; 31.82; 36.27; 36.44; 52.80; 123.27; 124.55; 127.20; 129.67; 129.89; 134.57; 141.08; 141.85; 171.66; 171.71; 173.86; 174.03

### Example 7

### Synthesis of O,O′-dibutanonyl-L-DOPA-DHA-amide (compound 7) and butanonyl-L-DOPA-di-DHA derivative (compounds 7A and 7B)

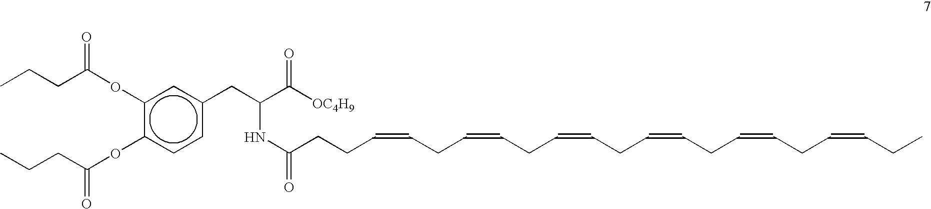

L-Dopa was converted to the n-butyl ester by n-butanol and thionyl chloride

Subsequently, the fatty acid DHA was converted, at a temperature of −10° C., to the mixed “active ester” by means of chloroformic acid isobutyl ester and reacted with the L-Dopa-n-butyl ester. Due to the further reaction with butyryl chloride, two products which were separated by chromatography were obtained. The polar compound was obtained with a yield of 23% and was the desired target compound according to the NMR analysis. The less polar compound was obtained with a yield of 34% and according to the NMR it contains two DHA-fatty acid residues. In this context, it is not clear whether the second DHA-fatty acid is bound to the phenol group in the para position or to the phenol group in the meta position

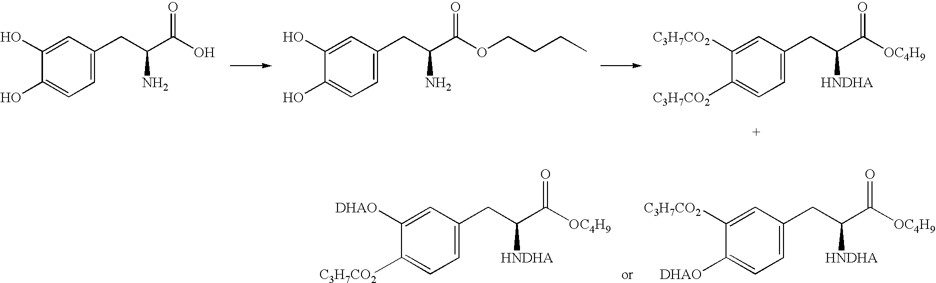

### Reaction Step 1:

2 g of 2,3-dihydroxyphenylalanine were dissolved in 10 ml of n-butanol and slowly mixed with 0.5 ml of thionyl chloride. Subsequently, stirring was continued for another 2 hrs at room temperature. Afterwards, the reaction solution was mixed with 50 ml of 2N HCl solution and with 50 ml of acetic acid ethyl ester. The aqueous phase was subsequently extracted another 3 times with acetic acid ethyl ester. The aqueous phase was mixed with K₂CO₃ until CO₂ formation could no longer be observed. Another 3 extractions with acetic acid ethyl ester were carried out. The combined organic phases were dried over Na₂SO₄ and concentrated. 0.9 g of 2,3-dihydroxyphenylalanine-n-butyl ester were obtained.

### Reaction Step 2:

1.35 g of DHA were dissolved in 30 ml of dichloromethane and cooled to a temperature of −10° C. Subsequently, 604 μl of chloroformic acid isobutyl ester and 634 μl of triethylamine were added and the mixture was stirred for 10 minutes at said temperature. To said mixture, 0.90 g of 2,3-dihydroxyphenylalanine-n-butyl ester dissolved in a mixture of 5 ml of dichloromethane and 5 ml of tetrahydrofurane were added dropwise for approx. 2 min. The reaction solution was stirred for another 2 hrs at 0 C. Subsequently, the mixture was cooled to −10° C. 1087 μl of triethylamine and 817 μl of butyric acid chloride were added. Upon addition, the mixture was slowly heated to room temperature and stirring was continued for another 2 hrs. Subsequently, the organic phase was extracted with water. After drying and concentrating, chromatography on silica gel (400 ml) was performed. (acetic acid ethyl ester 1: hexane 5) 0.63 g 1 (23%) and 1.13 g 2 (34%) were obtained.

NMR analysis regarding compound 7:

¹H-NMR (400.13 MHz, CD₃OD):

δ [ppm]=0.7-1.11 m (12H); 1.33 m (2H); 1.55 m (2H); 1.74 m (4H); 2.05 m (2H); 2.24 m (2H); 2.36 m (2H); 2.48 m (4H); 2.83 m (10H); 3.09 m (2H); 4.08 m (2H); 4.83 m (1H); 5.36 m (12H); 6.05 m (1H); 6.86-7.12 m (3H)

¹³C-NMR; DEPT; COSY ¹H/¹³C (100.625 MHz, CD₃OD):

δ [ppm]=13.49; 14.12; 18.28; 18.66; 18.92; 20.43; 23.06; 25.43; 25.53; 30.33; 35.76; 36.00; 37.15; 52.82; 65.40; 123.23; 124.41; 126.94; 126.97; 127.97; 127.78; 128.00; 128.03; 128.11; 128.14; 128.44; 129.22; 131.88; 134.50; 141.07; 141.90; 170.55; 170.64; 171.30; 171.87

### HPLC (Purity):

94.2% (230 nm-DAD); solvent: heptane/acetic acid ethyl ester 90/10 iso; R_{f}=5.07 min

NMR analysis regarding compounds 7A and 7B:

¹H-NMR (400.13 MHz, CD₃OD):

δ [ppm]=0.7-1.11 m (12H); 1.32 m (2H); 1.58 m (2H); 1.74 m (2H); 2.07 m (4H); 2.25 m (2H); 2.39 m (2H); 2.49 m (4H); 2.57 m (2H); 2.84 m (20H); 3.11 m (2H); 4.10 m (2H); 4.85 m (1H); 5.38 m (24H); 6.01 m (1H); 6.86-7.12 m (3H)

¹³C-NMR; DEPT; COSY¹H/¹³C (100.625 MHz, CD₃OD):

δ [ppm]=13.52; 13.56; 14.17; 18.53; 18.98; 20.48; 22.58; 23.10; 25.48; 25.58; 30.38; 33.86; 35.84; 36.06; 37.21; 52.86; 65.48; 123.24; 123.29; 124.40; 124.47; 126.96; 126.98; 127.02; 127.34; 127.79; 127.83; 127.98; 128.05; 128.10; 128.17; 128.19; 128.20; 128.23; 128.38; 128.50; 128.51; 129.29; 129.74: 131.94; 134.60; 141.07; 141.91; 170.19; 170.57; 171.34; 171.91

### HPLC (Purity):

97.8% (230 nm-DAD); solvent: heptane/acetic acid ethyl ester 90/10 iso; R_{f}=4.29 min

### Example 8

### Synthesis of L-DOPA-diacetyl acid derivative (compounds 8A and 8B) and L-DOPA-triacetyl acid derivative (compound 8)

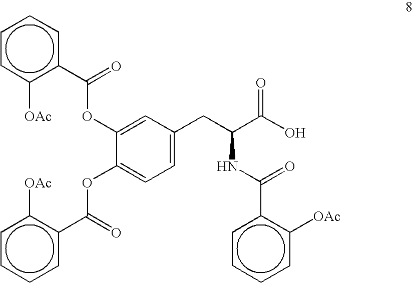

L-Dopa was reacted with an excess of acetylsalicylic acid chloride. After chromatographic purification it was found that the isolated product was not a pure substance, but a mixture of the triacylated compound 8 and the diacylated compounds (8A and 8B). The NMR spectrum and the HPLC chromatogram show that the target compound is present in a ratio of 2:1:1 with respect to the diacylated components.

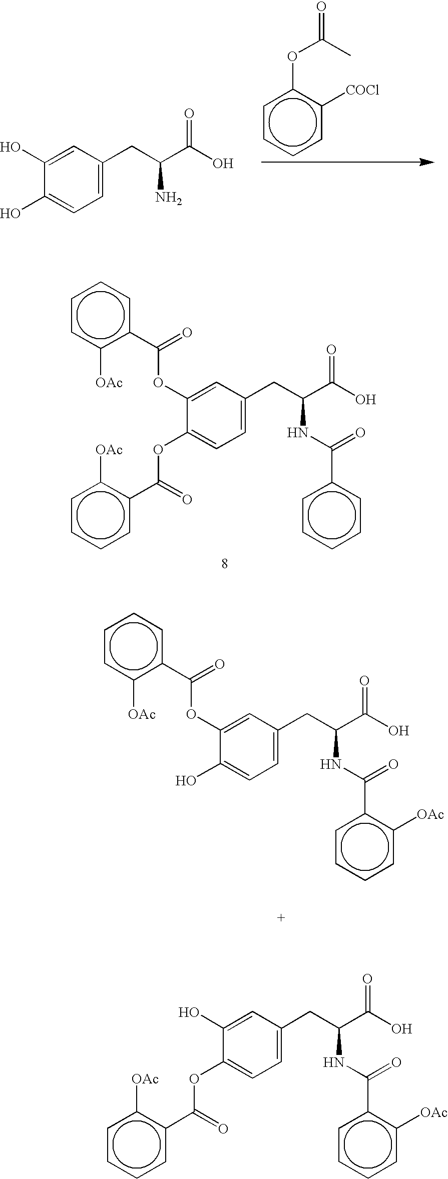

Reaction Mixture:

| Quantity | | Compound | Molar Weight | mmol |
|---|---|---|---|---|
| 1.0 | g | L-DOPA | 197.19 | 5.0 |
| 3.97 | g | acetylsalicylic acid chloride | 198.61 | 20.0 |
| 35 | ml | NaHCO₃ solution | | |
| 45 | ml | acetone nitrile | | |
| 3.36 | g | sodium hydrogen carbonate | 84.01 | 40 |

### Realization

1.0 g of L-DOPA with 3.36 g of sodium hydrogen carbonate were placed in 35 ml of NaHCO₃ solution and 25 ml of acetone nitrite. The suspension was degassed under water jet vacuum and deaerated with argon. A solution of 3.97 g of acetylsalicylic acid chloride in 20 ml of acetone nitrile was added dropwise within a period of 45 minutes. The mixture was stirred overnight at room temperature. After the addition of 130 ml of ethyl acetate and 50 ml of water, the phases were acidified with diluted hydrochloric acid under vigorous stirring. The phases were separated and the organic phase was washed with saturated NaCl solution, dried over sodium sulfate and concentrated on the rotary evaporator. The residue was chromatographed on 300 ml silica gel 60 (eluent: methylene chloride/ethyl acetate/formic acid=8:2:0.075 to 4:6:0.075). The product fraction was concentrated on the rotary evaporator and the remaining solvent components were removed under high vacuum. 1.05 g (30.7%) of an amorphous foam were obtained.

### TLC Conditions

Solvent: CH₂Cl₂/ethyl acetate/HCO₂H=5:5:0.075; detection: UV; 12 chamber or respectively KMnO₄ solution R_{f}=0.32

### HPLC—Scatter Detector

Rₜ=4.25 min (20.7%); 4.48 min (21.8%); 5.25 min (46.9%)

NMR analysis regarding compound 8:

¹H-NMR (400.13 MHz, CD₃OD):

δ [ppm]=2.14 s (3H); 2.21 s (3H); 2.25 s (3H); 3.27 m (1H); 3.40 m (1H); 5.09 m (1H); 6.80-8.2 m (15H)

¹³C-NMR (100.625 MHz, CD₃OD):

δ [ppm]=20.40; 20.75; 20.94; 36.39; 53.55; 111.19; 117.85; 119.63; 121.51; 123.33; 123.84; 124.59; 125.86; 125.98; 126.10; 127.20; 127.83; 128.15; 130.13; 132.42; 134.71; 135.14; 136.71; 140.65; 141.24; 142.03; 148.33; 161.87; 165.09; 167.60; 168.32; 168.97; 169.01; 170.00

### Description of Pharmacological Effects

Surprisingly, it has been found that L-DOPA derivatives (referred to as compound 1, compound 2, compound 3 in the following description) and also salts of said compounds can be used according to the invention for the prophylaxis and/or the treatment of, for example, Parkinson's disease and other movement disorders (secondary Parkinson syndrome).

In order to prove the efficacy of the compounds, experiments were performed to test the effects of said compounds on the concentration of dopamine, its metabolites dihydroxyphenylacetic acid (DOPAC), homovanillic acid (HVA) and 3-methoxytyramine (3-MT) as well as of 5-hydroxytryptamine (5-HT, also known as serotonin) and its metabolite 5-hydroxyindoleacetic acid (5-HIAA) in parts of the brain (striatum) and in the blood plasma of the rat.

The rats were pretreated with benserazide, an inhibitor of the aromatic amino acid decarboxylase, in order to reduce the degradation of the test compounds in the blood and to thus provide sufficient concentration of dopamine and α-lipoic acid in the brain. After 30 minutes, L-DOPA (standard therapy for Parkinson's disease) or compound 1, compound 2, compound 3 in doses equivalent to L-DOPA were injected into the peritoneum (intraperitoneal, ip). After a further 90 minutes, blood and brain tissue (striatum) were extracted. A lower dose (25 mg/kg body weight) and a higher dose (50 mg/kg body weight) of L-DOPA were selected.

TABLE 5
Summarizing statistics of the effects of L-DOPA, compound 1,
compound 2, compound 3 on the concentration of dopamine, its
metabolites Dopac, HVA and 3-MT as well as on serotonin (5-HT) and
its metabolite 5-HIAA in the brain (striatum) of the rat. The values are
given in pg per mg striatum.

| | | | | | | |
|---|---|---|---|---|---|---|
| Treatment | Dopamine | Dopac | HVA | 3-MT | 5-HT | 5-HIAA |

benserazide/solutol (n = 10)

| | | | | | | |
|---|---|---|---|---|---|---|
| mean value | 10170 | 1379 | 653 | 330 | 344 | 566 |
| standard | 687 | 173 | 88 | 101 | 81 | 122 |
| deviation | | | | | | |

benserazide + L-DOPA (25 mg/kg) (n = 6)

| | | | | | | |
|---|---|---|---|---|---|---|
| MV | 10108 | 3017 | 1752 | 312 | 365 | 711 |
| SD | 1193 | 982 | 220 | 51 | 73 | 113 |
| t-test | 0.4563 | 0.0001 | 0.0000 | 0.3591 | 0.3237 | 0.0217 |
| significance | | ** | ** | | | * |

benserazide + L-DOPA (50 mg/kg) (n = 6)

| | | | | | | |
|---|---|---|---|---|---|---|
| MV | 11844 | 9159 | 4550 | 259 | 366 | 593 |
| SD | 1676 | 204 | 695 | 55 | 60 | 74 |
| t-test | 0.0179 | 0.0000 | 0.0000 | 0.0810 | 0.3085 | 0.3360 |
| significance | * | ** | ** | | | |

benserazide + 25 mg/kg L-DOPA eq. compound 3 (n = 2)

| | | | | | | |
|---|---|---|---|---|---|---|
| MV | 10539 | 1508 | 1042 | 292 | 340 | 620 |
| SD | 699 | 116 | 167 | 21 | 25 | 43 |
| t-test | 0.2806 | 0.1881 | 0.0007 | 0.3192 | 0.4738 | 0.2912 |
| significance | | | ** | | | |

benserazide + 50 mg/kg L-DOPA eq. compound 3 (n = 3)

| | | | | | | |
|---|---|---|---|---|---|---|
| MV | 11488 | 1788 | 1244 | 237 | 278 | 490 |
| SD | 451 | 110 | 18 | 34 | 70 | 81 |
| t-test | 0.0105 | 0.0022 | 0.0000 | 0.0888 | 0.1363 | 0.1848 |
| significance | * | ** | ** | | | |

benserazide + 25 mg/kg L-DOPA-eq. compound 2 (n = 4)

| | | | | | | |
|---|---|---|---|---|---|---|
| MV | 10512 | 6127 | 1577 | 267 | 438 | 780 |
| SD | 1288 | 391 | 143 | 28 | 40 | 96 |
| t-test | 0.2980 | 0.0000 | 0.0000 | 0.1399 | 0.0343 | 0.0065 |
| significance | | ** | ** | | * | ** |

benserazide + 50 mg/kg L-DOPA-eq. compound 2 (n = 5)

| | | | | | | |
|---|---|---|---|---|---|---|
| MV | 11485 | 5697 | 1742 | 176 | 448 | 782 |
| SD | 1160 | 793 | 244 | 23 | 66 | 49 |
| t-test | 0.0188 | 0.0000 | 0.0000 | 0.0081 | 0.0220 | 0.0039 |
| significance | * | ** | ** | ** | * | ** |

benserazide + 25 mg/kg L-DOPA-eq. compound 1 (n = 7)

| | | | | | | |
|---|---|---|---|---|---|---|
| MV | 11244 | 4532 | 2567 | 308 | 355 | 706 |
| SD | 1460 | 702 | 813 | 47 | 76 | 130 |
| t-test | 0.054 | 0.0000 | 0.0000 | 0.31 | 0.40 | 0.024 |
| significance | | ** | ** | | | * |

benserazide + 50 mg/kg L-DOPA-eq. compound 1 (n = 6)

| | | | | | | |
|---|---|---|---|---|---|---|
| MV | 11695 | 9549 | 4314 | 194 | 411 | 764 |
| SD | 2877 | 1328 | 978 | 33 | 82 | 113 |
| t-test | 0.1028 | 0.0000 | 0.0000 | 0.0087 | 0.0865 | 0.0044 |
| significance | | ** | ** | ** | | ** |

vs control.
* p < 0.05
** p < 0.01

Table 5 shows that low doses of L-DOPA and compounds 1, 2 and 3 do not lead to an increase in the concentrations of dopamine in a part of the brain (striatum). However, the metabolites DOPAC and HVA are increased after administration of L-DOPA, compound 3 (only HVA), compound 2 and compound 1. This indicates that the conversion of the chemical messenger dopamine in the nerve cells increases due to the treatment. Furthermore, the results show that in the examined part of the brain the chemical messenger dopamine is formed in increased quantities from L-DOPA, compound 3 (some), compound 2 and compound 1. The higher doses of L-DOPA, compound 3 and 2 led to an increase in the concentrations of dopamine in the examined part of the brain. Also, the concentrations of the metabolites of dopamine, namely DOPAC and HVA were increased after administration of L-DOPA, compounds 1, 2 and 3; in fact, in almost all cases the increase was superior to that observed after administration of the lower dose. This indicates that all the substances used lead to an increase in the conversion of dopamine in the dopamine-containing nerve cells. Moreover, the results suggest that the compounds 1, 2 and 3 are capable of compensating for deficits of dopamine in nerve cells containing dopamine, a fact which is also known from L-DOPA. Such deficits are the known cause for movement disorders in Parkinson's disease. Interestingly, compound 2 also increased the concentration of 5-HT, even though it is assumed that in Parkinson's disease nerve cells in the brain containing 5-HT act as substitutes for the destroyed dopamine-containing nerve cells.

TABLE 6
Summarizing statistics of the effects of the administration of L-DOPA
and equimolar doses of compound 1 and compound 2 on the
concentration of dopamine and its metabolites Dopac, HVA and 3-MT
as well as of 5-HT and its metabolite 5-HIAA in the blood plasma of rats.
The values are given in pg per ml plasma.

| | | | | | | |
|---|---|---|---|---|---|---|
| Treatment | Dopamine | Dopac | HVA | 3-MT | 5-HT | 5-HIAA |

benserazide/solutol

| | | | | | | |
|---|---|---|---|---|---|---|
| mean value | 4511 | 11099 | 10840 | 4804 | 14924 | 8701 |
| standard | 207 | 2774 | 981 | 743 | 40 | 2222 |
| deviation | | | | | | |

benserazide + L-DOPA (25 mg/kg body weight)

| | | | | | | |
|---|---|---|---|---|---|---|
| MV | 20437 | 20833 | 9592 | 11897 | 3089 | 18687 |
| SD | 1231 | 8565 | 10089 | 3677 | 1483 | 2881 |
| t-test | 2.77E−05 | 1.00E−01 | 4.35E−01 | 4.34E−02 | 1.58E−03 | 4.16E−03 |
| significance vs | ** | | | * | ** | ** |
| control | | | | | | |

benserazide + L-DOPA (50 mg/kg)

| | | | | | | |
|---|---|---|---|---|---|---|
| MV | 31519 | 38697 | 69329 | 14711 | 15108 | 11426 |
| SD | 1131 | 11973 | 10263 | 2932 | 5711 | 4714 |
| t-test | 2.45E−06 | 1.06E−02 | 6.55E−04 | 4.90E−03 | 4.87E−01 | 2.34E−01 |
| significance vs | ** | ** | ** | ** | | |
| control | | | | | | |

benserazide + compound 2 (25 mg/kg L-DOPA eq.)

| | | | | | | |
|---|---|---|---|---|---|---|
| MV | 23006 | 52963 | 44304 | 11480 | 7966 | 8374 |
| SD | 6486 | 17787 | 10944 | 1064 | 1819 | 2016 |
| t-test | 0.0044 | 0.0095 | 0.0033 | 0.0003 | 0.0058 | 0.4352 |
| significance vs | ** | ** | ** | ** | ** | |
| control | | | | | | |

benserazide + compound 2 (50 mg/kg L-DOPA eq.)

| | | | | | | |
|---|---|---|---|---|---|---|
| MV | 21919 | 57729 | 42184 | 6121 | 7494 | 12656 |
| SD | 1908 | 31791 | 8296 | 961 | 2762 | 4350 |
| t-test | 0.0002 | 0.0823 | 0.0052 | 0.0948 | 0.0262 | 0.1832 |
| significance vs | ** | | ** | | * | |
| control | | | | | | |

benserazide + compound 1 (25 mg/kg L-DOPA eq.)

| | | | | | | |
|---|---|---|---|---|---|---|
| MV | 29782 | 57257 | 16836 | 8563 | 8894 | 13852 |
| SD | 4276 | 8511 | 4903 | 7638 | 7244 | 2445 |
| t-test | 0.0002 | 0.0003 | 0.0826 | 0.2525 | 0.2145 | 0.0460 |
| significance vs | ** | ** | | | | * |
| control | | | | | | |

benserazide + compound 1 (50 mg/kg L-DOPA eq.)

| | | | | | | |
|---|---|---|---|---|---|---|
| MV | 22129 | 58266 | 71658 | 6288 | 12094 | 15367 |
| SD | 5705 | 29342 | 34535 | 2152 | 1930 | 2433 |
| t-test | 0.0031 | 0.0432 | 0.0338 | 0.2043 | 0.0829 | 0.0458 |
| significance vs | ** | * | * | | | * |
| control | | | | | | |

vs control.
* p < 0.05
** p < 0.01

The values obtained from blood plasma analysis (table 6) prove that dopamine is formed from L-DOPA both after the administration of the low and of the higher doses. Dopamine is also formed from compounds 1 and 2 upon administration of both doses. Thus, at first the compounds are cleaved and subsequently dopamine is formed from the thus released L-DOPA. In most cases, the concentrations of the metabolites of dopamine, namely Dopac, HVA and 3-MT increase, too. These results confirm the assumption that dopamine is formed from all examined compounds in the blood.

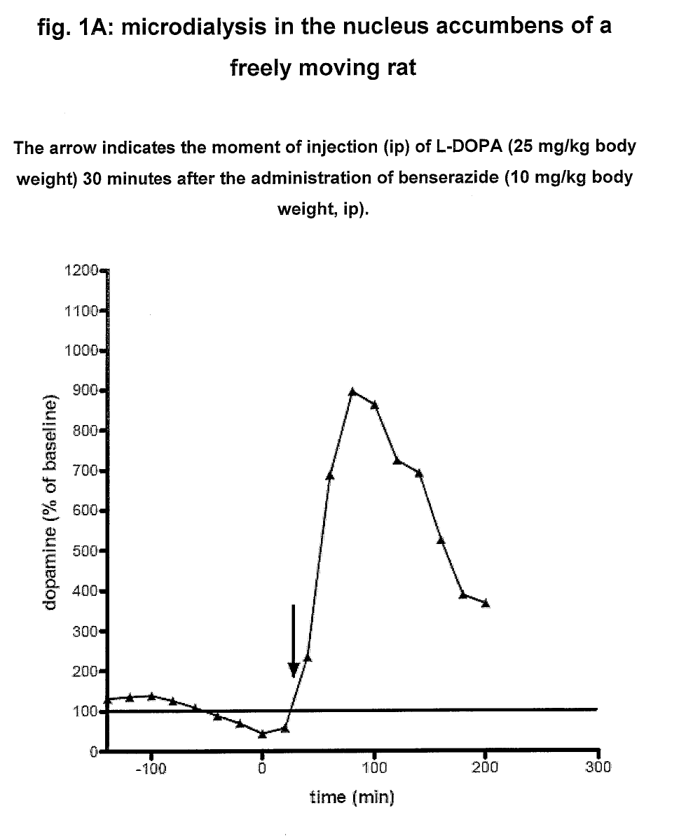

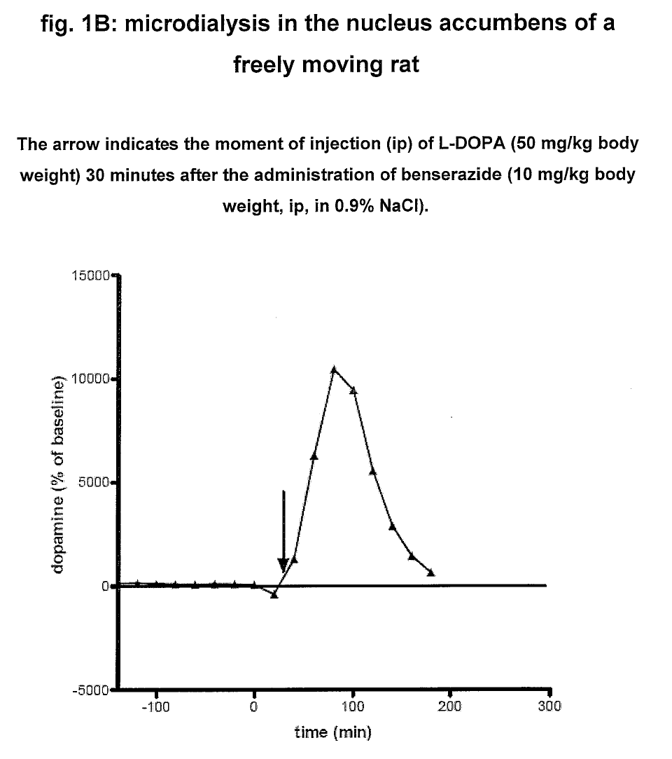

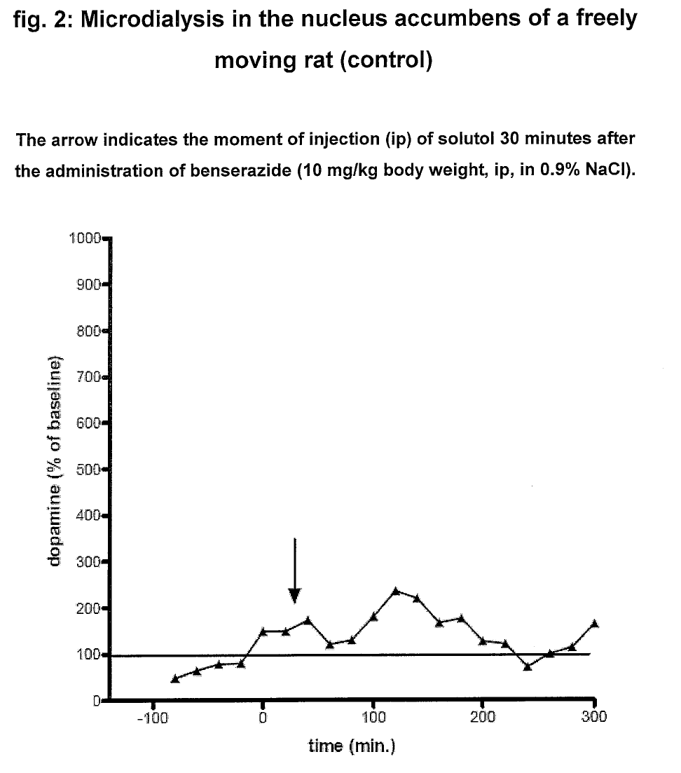

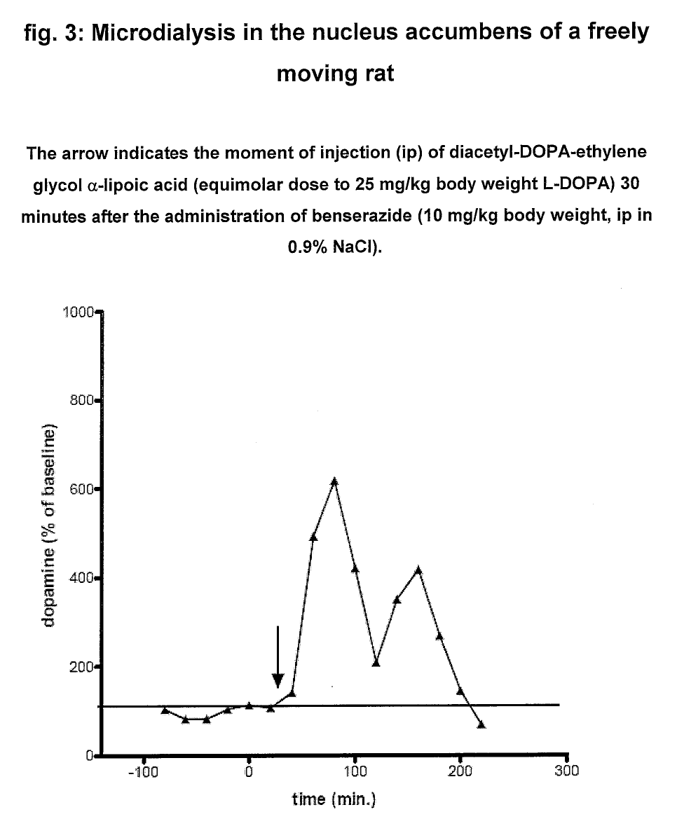

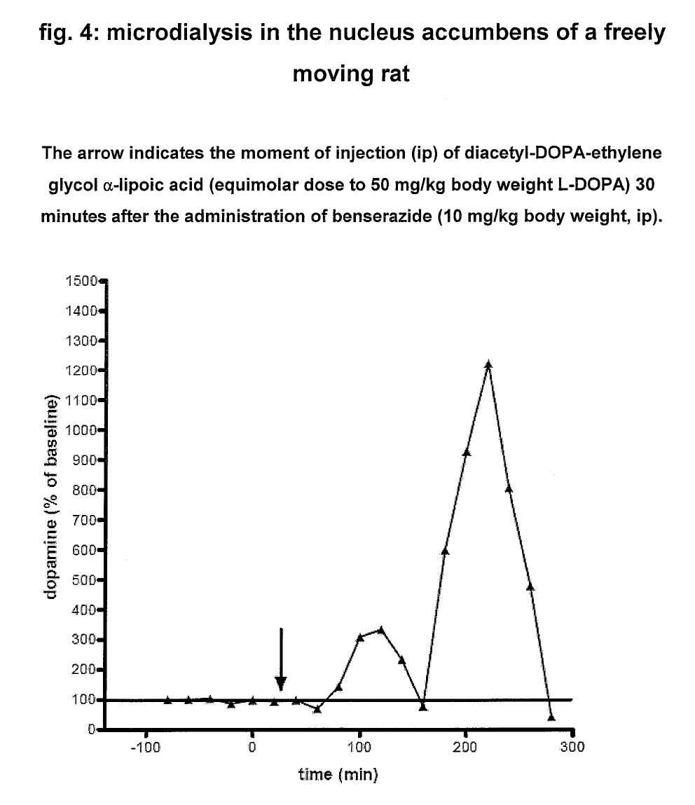

Furthermore, by the method of in vivo micro dialysis of the conscious and freely moving rat, experiments were carried out to test whether the production and release of dopamine in the nucleus accumbens, a part of the brain with dense innervation of dopamine-containing nerve cells, is increased upon administration of L-DOPA or compound 1. The advantage of said method consists in the fact that the time-course of the release of the messenger dopamine from the active nerve cells can be observed in the conscious and freely moving rat. As can be seen from FIGS. 1-4, dopamine is released in a dose-dependent manner. Upon administration of the higher dose of L-DOPA, the concentration of dopamine strongly increases and the concentrations are comparatively very high (FIG. 1). Said strong increase and the high concentrations are not desired since several degradation products of dopamine and the oxygen radicals formed during the degradation of dopamine have damaging effects on nerve cells. Compound 1 has a two-peak-maximum and a less steep increase as well as a longer lasting effect (FIGS. 3 and 4)

The Coupling of L-Dopa to α-Lipoic Acid has an Antioxidative Effect (α-Lipoic Acid binds harmful oxygen radicals and inactivates them). The toxic oxygen radicals which are formed in large quantities during the degradation of dopamine destroy dopaminergic nerve cells. They are the main reason for the death of dopaminergic nerve cells. Therefore, the short term high concentrations of dopamine after administration of L-DOPA are destructive. The results suggests that the advantageous α-lipoic acid is released from compound 1 in the vicinity of or directly within the dopaminergic nerve cells and can develop its protecting effect in situ, that is within the dopamine-containing nerve cells where the damaging oxygen radicals are formed. This results in the further loss of dopamine-containing nerve cells in the brain being slowed down or potentially even being stopped.

1. Compounds of the general formula I 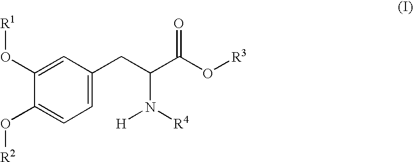
wherein
R¹ and R² represent, independently of each other, the following residues: —H, —R⁸, —R⁹, —CO—H, —CO—CH₃, —CO—C₂H₅, —CO—C₃H₇, —CO—C₄H₉, —CO—C₅H₁₁, —CO—C₆H₁₃, —CO—CH(CH₃)₂, —CO-cyclo-C₃H₅, —CO—CH₂—CH(CH₃)₂, —CO—CH(CH₃)—C₂H₅, —CO—C(CH₃)₃, —CO-cyclo-C₄H₇, —CO-cyclo-C₅H₉, —CO-cyclo-C₆H₁₁, —C≡CH, —C═C—CH₃, —CH₃, —C₂H₅, —C₃H₇, —CH(CH₃)₂, —C₄H₉, —CH₂—CH(CH₃)₂, —CH(CH₃)—C₂Hs, —C(CH₃)₃, —C₅H₁, —CH(CH₃)—C₃H₇, —CH₂—CH(CH₃)—C₂H₅, —CH(CH₃)—CH(CH₃)₂, C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CF₃, —C₂F₅, —CH(CH₃)—CH(CH₃)—C₂H₅, —CH₂—CH(CH₃)CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —CH₂—CH═CH—CH₃, —CH═CH₂, —CH₂—CH═CH₂, —CH═CH—CH₃, -cyclo-C₃H₅, -cyclo-C₄H₇, -cyclo-C₅H₉, -cyclo-C₆H₁₁, 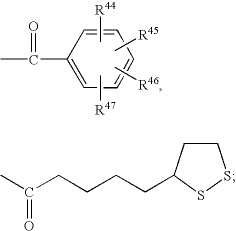
R³ represents a residue —CH₂CH₂O—R⁵, —H, —C≡CH, —C≡C—CH₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₇, —CH(CH₃)—C₃H₇, —CH₂—CH(CH₃)—C₂H₅, —CH(CH₃)—CH(CH₃)₂, —C(CH₃)₂C₂H₅, CH₂CH(CH₃)₃, —CH(C₂H₅)₂, —C₂H₄—CH(CH₃)₂, —C₆H₁₃, —C₃H₆—CH(CH₃)₂, —C₂H₄—CH(CH₃>C₂H₅, —CH(CH₃)—C₄H₉, —CH₂—CH(CH₃—C₃H₇, —CH(CH₃)—CH₂CH(CH₃)₂, —CF₃, —C₂F₅, —CH(CH₃)—CH(CH₃)—C₂H₅, —CH₂—CH(CH₃)—CH(CH₃)₂, CH₂—C(CH₃)₂—C₂H₅, —C(CH₃)₂—C₃H₇, <(CH₃)₂—CH(CH₃)₂, —C₂H₄—C(CH₃)₃, —CH(CH₃)<(CH₃)₃, —CH═CH₂, —CH₂—CH═CH₂, —C(CH₃)═CH₂, —CH═CH—CH₃, —C₂H₄CH═CH₂, —CH₂—CH═CH—CH₃, —CH═CH₂, —CH₂—CH═CH₂, —CH═CH—CH₃, -cyclo-C₃H₅, -cyclo-C₄H₇, -cyclo-C₅H₉, -cyclo-C₆H₁₁,
R⁴ and R⁵ represent, independently of each other, a group —CO—R⁶ or —CO—R⁷ or —H, wherein R³ and R⁴ do not at the same time represent —H; wherein R³ represents —CH₉CH₂O—R⁵ if R⁴ is hydrogen; and
R⁶ and R⁷ represent, independently of each other, the following residues:
—R¹⁰; —R¹¹; a linear saturated alkyl chain with 2-25 carbon atoms; a branched saturated alkyl chain with 2-25 carbon atoms; a branched or unbranched alkenyl chain with 2-25 carbon atoms; a branched or unbranched alkinyl chain with 2-25 carbon atoms; a polyunsaturated branched or unbranched alkenyl chain with 2-25 carbon atoms; a polyunsaturated branched or unbranched alkinyl chain with 2-25 carbon atoms; a polyunsaturated branched or unbranched alkeninyl chain with 2-25 carbon atoms; a branched or unbranched alkyl chain with 2-25 carbon atoms comprising a carbocycle or a heterocycle; a branched or unbranched alkyl chain with 2-25 carbon atoms comprising one or more hydroxy groups, alkoxy groups, thio groups, mercapto groups, amino groups, halogen groups, carbonyl groups, carboxyl groups and/or nitro groups;
R⁸, R⁹, R¹⁰ and R¹¹ represent, independently of each other, the following residues: —CH₂R¹², —CHR¹³R¹⁴, —CR¹⁵R¹⁶R¹⁷, CR¹⁸R¹⁹R²⁰, —CH₂—CHR²¹R²², —CR²³R²⁴—CR²⁵R²⁶R²⁷, —CR²⁸R²⁹—CR³⁰R³¹—CR³²R³³R³⁴,
—CR³⁵R³⁶—CR³⁷R³⁸—CR³⁹R⁴⁰—CR⁴¹R⁴²R⁴³; alkyl groups with 2-25 carbon atoms, which groups are substituted with one or more of the residues R¹² to R⁴³, alkenyl groups with 2-25 carbon atoms, which groups are substituted with one or more of the residues R¹² to R⁴³;
alkinyl groups with 2-25 carbon atoms, which groups are substituted with one or more of the residues R¹² to R⁴³, alkoxy groups with 2-25 carbon atoms, which groups are substituted with one or more of the residues R¹² to R⁴³, aryl groups with 2-25 carbon atoms, which groups are substituted with one or more of the residues R¹² to R⁴³, heteroaryl groups with 2-25 carbon atoms, which groups are substituted with one or more of the residues R¹² to R⁴³, heterocyclyl groups with 2-25 carbon atom s, which groups are substituted with one or more of the residues R¹² to R⁴³,
R¹²-R⁴⁷ represent, independently of each other, the following residues:
—H, —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂Hs, —SC₃H₇, —S-cyclo-C₃H₅, —SCH(CH₃)₂, —SC(CH₃)₃, —NO₂, —F, —Cl, —Br, I, —N₃, CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COCN, —COOCH₃, —COOC₂H₅, —COOC₃H₇, —COO-cyclo-C₃H₅, —COOCH(CH₃)₂, COOC(CH₃)₃, —OOCCH₃, —OOC—C₂H₅, OOCC₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CON(CH₃)₂, —CON(C₂H₅)₂, —CON(C₃H₇)₂, —CON(cyclo-C₃H₅)₂, —NH₂, —NHCH₃, —NHC₂H₅, —NHC₃H₇, —NH(cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SOCH(CH₃)₂, —SOC(CH₃)₃, —SO₂CH₃, —SO₂C₂H₅, —SO₂C₃H₇, —SO₂Cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —OCF₃, —OC₂F₅, —O—COOCH₃, —O—COOC₂H₅, —O—COOC₃H₇, —O—COO-cyclo-C₃H₅, —OCOOCH(CH₃)₂, —O—COOC(CH₃)₃, —NH—CO—NH₂, —NH—C(═NH)—NH₂, —O—CO—NH₂, —O—CO—NHCH₃, —O—CO—NHC₂H₅, —O—CO—NHC₃H₇, —O—CONH-cyclo-C₃H₅, —O—CO—N(CH₃)₂, —O—CO—N(C₂H₅)₂, —O—CO—N(C₃H₇)₂, —O—CO—OCH₃, —O—CO—OC₂H₅, —O—CO—OC₃H₇, —O—CO₄-cyclo-C₃H₅, —O—CO—OCH(CH₃)₂, —O—CO—OC(CH₃)₃, —CH₂F, —CH₂F, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, -Ph, —CH₂-Ph, —CPh₃, —CH═CH₂, —CH₂—CH═CH₂, —C(CH₃)═CH₂, —CH═CH—CH₃, —C₂H₄—CH═CH₂, CH═C(CH₃)₂, —C≡CH, —C≡C—CH₃, —CH₂—C≡CH; as well as
pharmacologically acceptable salts, solvates, hydrates, complex compounds, enantiomers, diastereomers and racemates of the aforementioned compounds.

2. Compound according to claim 1 of the general formula (VI): 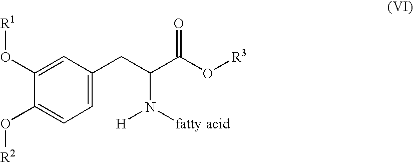
wherein fatty acid represents the residue —CO—R⁶ and the residues R¹, R² and R³ have the meaning indicated in claim 1 and
R⁶ represents the following residues: a linear saturated alkyl chain with 2-25 carbon atoms, a branched saturated alkyl chain with 2-25 carbon atoms, a branched or unbranched alkenyl chain with 2-25 carbon atoms, a branched or unbranched alkinyl chain with 2-25 carbon atoms, a polyunsaturated branched or unbranched alkenyl chain 2-25 carbon atoms, a polyunsaturated branched or unbranched alkinyl chain with 2-25 carbon atoms, a polyunsaturated branched or unbranched alkeninyl chain with 2-25 carbon atoms, a branched or unbranched alkyl chain with 2-25 carbon atoms comprising a carbocycle or a heterocycle, a branched or unbranched alkyl chain with 2-25 carbon atoms comprising one or more hydroxy groups, thio groups and/or mercapto groups.

3. Compound according to claim 1 of the general formula (VII): 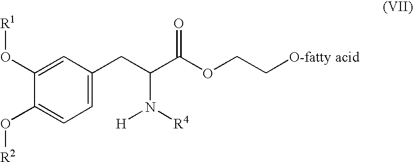
wherein fatty acid represents the residue —CO—R⁷ and the residues R¹, R², R³ and R⁷ have the same meaning as indicated in claim 1.

4. Compounds according to any one of claims 1-3, wherein said compounds have S-configuration at the carbon atom 2 of the propionic acid chain.

5. Compounds according to claim 1, wherein R⁴ or R⁵ represent hydrogen.

6. Compounds according to any one of claims 1-3, wherein R⁶ and R⁷ represent, independently of each other, a branched or unbranched alkyl chain with 5-9 carbon atoms, with said chain comprising one or more hydroxy groups, alkoxy groups, thio groups, mercapto groups, amino groups, halogen groups, carbonyl groups, carboxyl groups and/or nitro groups.

7. Compounds according to any one of claims 1-3, wherein the branched or unbranched, substituted or unsubstituted and saturated or unsaturated carbon residues of R⁶ and R⁷ comprise, independently of each other, 5 to 24 carbon atoms.

8. Compounds according to claim 7, wherein the branched or unbranched, substituted or unsubstituted and saturated or unsaturated carbon residues of R⁶ and R⁷ comprise, independently of each other, 7 to 23 carbon atoms.

9. Compounds according to claim 8, wherein the branched or unbranched, substituted or unsubstituted and saturated or unsaturated carbon residues of R⁶ and R⁷ comprise, independently of each other, 9 to 22 carbon atoms.

10. Compounds according to any one of claims 1-3, wherein R⁴ and R⁵, independently of each other, represent the following groups: dodecanoyl, hexadecanoyl, octadecanoyl, eicosanoyl, docosanoyl, tetracosanoyl, cis-9-tetradecenoyl, cis-9-hexadecenoyl, cis-6-octadecenoyl, cis-9-octadecenoyl, cis-11-octadecenoyl, cis-9-eicosenoyl, cis-11-eicosenoyl, cis-13-docosenoyl, cis-15-tetracosenoyl, 9,12-octadecadienoyl, 6,9,12-octadecatrienoyl, 8,11,14-eicosatrienoyl, 5,8,11,14-eicosatetraenoyl, 7,10,13,16-docosatetraenoyl, 4,7,10,13,16-docosapentaenoyl, 9,12,15-octadecatrienoyl, 6,9,12,15-octadecatetraenoyl, 8,1,14,17-eicosatetraenoyl, 5,8,11,14,17-eicosapentaenoyl, 7,10,13,16,19-docosapentaenoyl, 4,7,10,13,16,19-docosahexaenoyl, 5,8,11-eicosatrienoyl, 1,2-dithiolane-3-pentanoyl, 6,8-dithianeoctanoyl, docosaheptadecanoyl, eleostearoyl, calendoyl, catalpoyl, taxoleoyl, pinolenoyl, sciadonoyl, retinoyl, 14-methylpentadecanoyl, pristanoyl, phytanoyl, 11,12-methyleneoctadecanoyl, 9,10-methylenehexadecanoyl, 9,10-epoxystearoyl, 9,10-epoxyoctadec-12-enoyl, 6-octadecynoyl, t11-octadecen-9-ynoyl, 9-octadecynoyl, 6-octadecen-9-ynoyl, t10-heptadecen-8-ynoyl, 9-octadecen-12-ynoyl, t7,t11-octadecadiene-9-ynoyl, t8,t10-octadecadiene-12-ynoyl, 5,8,11,14-eicosatetraynoyl, 2-hydroxytetracosanoyl, 2-hydroxy-15-tetracosenoyl, 12-hydroxy-9-octadecenoyl and 14-hydroxy-11-eicosenoyl.

11. Compounds according to claim 10 wherein R⁴ and R⁵, independently of each other, represent the following groups: 9,12-octadecadienoyl, 6,9,12-octadecatrienoyl, 8,11,14-eicosatrienoyl, 5,8,11,14-eicosatetraenoyl, 9,12,15-octadecatrienoyl, 6,9,12,15-octadecatetraenoyl, 8,11,14,17-eicosatetraenoyl, 5,8,11,14,17-eicosapentaenoyl, 7,10,13,16,19-docosapentaenoyl, 4,7,10,13,16,19-docosahexaenoyl, 5,8,11-eicosatrienoyl, 1,2-dithiolane-3-pentanoyl and 6,8-dithianeoctanoyl.

12. Compounds according to any one of claims 1-3 for use as pharmacologically active substance.

13. Use of the compounds according to any one of claims 1-3 for the treatment and/or prophylaxis of movement disorders, early-onset drug-induced dyskinesias, akathisia, parkinsonian features, rigidity, tremor, extrapyramidal dysfunctions, segmented dystonias, generalized dystonias, drug-induced extrapyramidal symptoms, different forms of parkinsonian syndromes, endogenous parkinsonian syndrome, atherosclerotic parkinsonian syndrome, postencephalitic parkinsonian syndrome, drug-induced parkinsonian syndrome, neurodegenerative diseases, Alzheimer's disease, Parkinson's disease, hemiparkinson-hemiatrophy, parkinsonian syndrome, Lewy body disease, frontotemporal dementia, Lytico-Bodig disease (parkinsonism/dementia/amyotrophic lateral sclerosis), striatonigral degeneration, Shy-Drager syndrome, sporadic olivopontocerebellar degeneration, progressive atrophy of the globus pallidus, progressive supranuclear palsy, Hallervorden-Spatz disease, Huntington's disease, X-linked dystonia-parkinsonism (Lubag), mitochondrial cytopathy with striatal necrosis, neuroacanthocytosis, restless legs syndrome, Wilson's disease.

14. Pharmaceutical composition comprising at least one compound according to the general formula (I) of claim 1 and/or pharmacologically acceptable salts thereof and a pharmacologically acceptable carrier, adjuvant and/or diluents.

15. Pharmaceutical composition according to claim 14 in form of drops, mouth spray, nose spray, pills, tablets, film coated tablets, multi-layered tablets, suppositories, gels, ointments, syrups, inhalation powders, granulates, emulsions, dispersions, microcapsules, capsules, powders or solutions for injection.

16. Pharmaceutical composition according to claim 14 suitable for inhalation or intravenous, intraperitoneal, intramuscular, subcutaneous, mucocutaneous, oral, rectal, transdermal, topical, buccal, intradermal, intragastric, intracutaneous, intranasal, intrabuccal, percutaneous or sublingual administration.

17. Pharmaceutical composition according to any one of claims 14-16, wherein an additional pharmacological agent is present which is suitable for the treatment and/or prophylaxis of movement disorders, neurodegenerative diseases, Alzheimer's disease, Parkinson's disease, hemiparkinson-hemiatrophy, parkinsonian syndrome, Lewy body disease, frontotemporal dementia, Lytico-Bodig disease (parkinsonism/dementia/amyotrophic lateral sclerosis), striatonigral degeneration, Shy-Drager syndrome, sporadic olivopontocerebellar degeneration, progressive atrophy of the globus pallidus, progressive supranuclear palsy, Hallervorden-Spatz disease, Huntington's disease, X-linked dystonia-parkinsonism (Lubag), mitochondrial cytopathy with striatal necrosis, neuroacanthocytosis, restless legs syndrome, Wilson's disease.

18. Pharmaceutical composition according to claim 17, wherein the additional pharmacological agent is selected from the group comprising: bromocriptine, cabergoline, lisuride, dihydroergocriptine, dopamine agonists, entacapone, ropinirole, pramipexole, pergolide mesylate, pergolide, NMDA glutamate receptor antagonists, amantadine, budipine, monoamine oxidase B inhibitors, selegiline, catechol-O-methyltransferase inhibitors, anticholinergics, benzatropine, biperiden, bomaprine, procyclidine, trihexyphenidyl, antioxidants, vitamin C and vitamin E.
